(19) United States
(12) Patent Application Publication
(10) Pub. No.: US 2022/0157454 A1
(43) Pub. Date: May 19, 2022

(54) SYSTEM, METHOD, AND PROGRAM FOR PATIENTS WITH ERECTILE DYSFUNCTION

(71) Applicant: BIOMIMETICS SYMPATHIES. Inc, Tokyo (JP)

(72) Inventors: Hideki TANEMURA, Tokyo (JP); Koji KOIDE, Tokyo (JP); Ghazizadeh MOHAMMAD, Tokyo (JP); Yasuhiro NOSE, Tokyo (JP); Yoichiro NISHINO, Tokyo (JP); Chigusa SHIMOKAWA, Tokyo (JP); Kuo CHEN, Tokyo (JP)

(73) Assignee: BIOMIMETICS SYMPATHIES. Inc, Tokyo (JP)

(21) Appl. No.: 17/441,580

(22) PCT Filed: Mar. 27, 2020
(86) PCT No.: PCT/JP2020/013932

§ 371 Date: Sep. 21, 2021

(30) Foreign Application Priority Data
Mar. 27, 2019 (JP) 2019-059756
Mar. 25, 2020 (JP) 2020-053760

(51) Int. Cl. G16H 40/67; G16H 10/60; G16H 20/00

(52) U.S. Cl. CPC G16H 40/67; G16H 20/00; G16H 10/60

(57) ABSTRACT

Provided are a system and the like for effectively treating ED by enabling not only administration of a PDE5 inhibitor but also execution of psychotherapy and other therapies through an information network and an information processing terminal while minimizing a treatment directly undergone face-to-face with a doctor or a medical worker. Therefore, a system (100) for a patient with ED includes: a control device (1); an information processing terminal (3) to be used by the patient; a database (2) configured to store information (for example, diagnosis-and-treatment history) on the patient and information relating to an erectile dysfunction treatment (for example, treatment method, information on the PDE5 inhibitor, and information relating to psychotherapy); and an information processing terminal (4) to be used on a medical institution side (for example, by the doctor), and in the system (100), the control device (1) has a function of selecting information (for example, prescription) relating to a drug to be taken and information relating to a therapy (for example, cognitive behavioral therapy) for resolving the erectile dysfunction from the information on the patient and the information relating to the erectile dysfunction treatment.

### BACKGROUND OF THE INVENTION

### 1. Field of the Invention

The present invention relates to a treatment of erectile dysfunction (ED).

### 2. Description of the Related Art

ED is classified into three types based on its pathogenesis, namely, into organic ED, psychogenic ED, and mixed ED.

The psychogenic ED is ED caused by psychological factors, and its causes are roughly divided into real psychological factors and deep psychological factors. The real psychological factors are mental and physical stress and psychological factors in real daily life. The deep psychological factors are psychological causes in a deep part of one's mind, for example, early childhood experiences and sexual trauma. Meanwhile, the organic ED is ED caused by, for example, aging, lifestyle-related diseases, nervous system disorders, and surgical damage. The mixed ED is ED that occurs due to a mixture of a cause of psychogenic ED and a cause of organic ED.

It is known as a result of various researches that ED is mostly organic ED due to arteriosclerosis in the age group of 50 to 60 years, but is often psychogenic ED in the age group of 35 to 45 years, which is a so-called “generation trying to have a child.”

A PDE5 inhibitor is administered as the first choice for an ED treatment, but has various side effects and contraindications. In addition, when the PDE5 inhibitor cannot be used or when the administration is discontinued, there are limited options left for the next treatment method. It is also known that a dropout rate of taking the PDE5 inhibitor is high.

In contrast, for the psychogenic ED, a cure rate is higher when the PDE5 inhibitor and psychotherapy are used in combination than when only the PDE5 inhibitor is used. Therefore, for psychogenic ED, it is strongly recommended to use the PDE5 inhibitor in combination with psychotherapy.

In this case, the ED treatment closely relates to privacy, and there is a request for a patient to keep the fact that the patient is “undergoing ED treatment” as secret as possible. In connection therewith, many ED patients resist undergoing diagnosis by a doctor face-to-face at a specialized medical institution. In particular, in the treatment of the psychogenic ED, such resistance is anticipated to interfere with the treatment. Therefore, in the psychogenic ED, regardless of being strongly recommended, many patients do not desire a treatment of psychotherapy by a specialized doctor or a specialist, and a consultation rate is low as well.

Meanwhile, when contact can be made through an information terminal and an information network (for example, the Internet), medical examination or diagnosis by a doctor is required for a prescription or another purpose, but can be minimized, and hence psychological resistance of the ED patient is small.

However, a program or system suitable for performing an ED treatment through an information terminal and an information network (for example, the Internet) has not yet been proposed.

In particular, in the case of the psychogenic ED, it is recommended as described above to use in combination not only the administration of the PDE5 inhibitor but also the psychotherapy, for example, a cognitive behavioral therapy, but it has not yet been achieved to execute the psychotherapy suitable for the psychogenic ED through an information terminal and an information network (for example, the Internet).

As another related art, a program for a patient with nicotine addiction (so-called patient who is attempting to quit smoking) is provided (see, for example, JP 6339298 B1).

However, the above-mentioned related art (JP 6339298 B1) is a technology relating to a program for a patient who is attempting to quit smoking, and cannot be applied to a treatment for a patient with erectile dysfunction (ED).

### SUMMARY OF THE INVENTION

The present invention has been proposed in view of the above-mentioned problems of the related art, and has an object to provide a system, method, and program for effectively treating ED by enabling not only administration of a PDE5 inhibitor but also execution of psychotherapy (for example, cognitive behavioral therapy) and other therapies through an information network and an information processing terminal while minimizing direct consultation with a doctor or a medical worker for an ED treatment.

A system (100) for a patient with erectile dysfunction (ED) according to at least one embodiment of the present invention includes: a control device (1: a computer or a server device); an information processing terminal (3: an electronic device or another terminal device having an information processing function) to be used by the patient; a database (2) configured to store information (for example, diagnosis-and-treatment history information) on the patient and information relating to an erectile dysfunction treatment (for example, treatment method, information relating to a PDE5 inhibitor, and information relating to psychotherapy including a cognitive behavioral therapy); and an information processing terminal (4: an electronic device or another terminal device having an information processing function) to be used on a medical institution side (for example, by a doctor), and in the system (100), the control device (1) has a function of selecting information (for example, information on the cognitive behavioral therapy) relating to a therapy (for example, psychotherapy including the cognitive behavioral therapy) for resolving the erectile dysfunction based on the information on the patient and the information relating to the erectile dysfunction treatment.

Further, a method for an ED patient according to at least one embodiment of the present invention is a method of using an information processing system (100), for example, the above-mentioned system for a patient with erectile dysfunction, the method including: transmitting a situation relating to ED of the patient from a patient-side information processing terminal (3) to a control device (1); determining, by the control device (1), information relating to an ED treatment in response to the received situation of the patient; and transmitting the determined information relating to the ED treatment to the patient-side information processing terminal (3).

Still further, a program for a patient with ED according to at least one embodiment of the present invention is a program for executing the following processes comprising: transmitting a situation relating to the ED of the patient from a patient-side information processing terminal (3) to a control device (1); determining, by the control device (1), information relating to an ED treatment in response to the situation of the patient; and transmitting the determined information relating to the ED treatment to the patient-side information processing terminal (3).

According to at least one embodiment of the present invention having the above-mentioned configuration, the ED patient can contact a doctor or a medical institution through an information terminal (3) and an information network (10: for example, the Internet). Therefore, the ED patient is only required to contact the doctor at a minimum necessary frequency for the prescription or another purpose, and the psychological resistance of the ED patient to the treatment is reduced, to thereby reduce a probability of dropping out during the treatment.

Further, according to at least one embodiment of the present invention in which the ED treatment can be performed through the information terminal (3) and the information network (10: for example, the Internet), visits to the medical institution and treatments undergone face-to-face with the doctor may be reduced to the minimum necessary, to thereby satisfy the patient's request to keep the fact that the patient is “undergoing ED treatment” as secret as possible. This is extremely important in the ED treatment, which closely relates to privacy, and can greatly reduce the ED patient's sense of resistance to undergoing the treatment face-to-face with the doctor at a specialized medical institution. Then, the degree of resistance felt by the patient is lowered, and a consultation rate is expected to be improved.

In regard to the ED treatment, it is desired even for the organic ED or the mixed ED to use in combination not only the administration of the PDE5 inhibitor but also the psychotherapy (for example, cognitive behavioral therapy) as strongly recommended by specialists. According to at least one embodiment of the present invention, the psychotherapy (for example, cognitive behavioral therapy) can be executed when the ED treatment is performed through the information terminal (3) and the information network (10: for example, the Internet).

It is to be understood that, according to at least one embodiment of the present invention, the treatment involving the administration of the PDE5 inhibitor can also be performed (in combination with the psychotherapy, for example, cognitive behavioral therapy) by, for example, a procedure for issuing a prescription for the PDE5 inhibitor.

The ED is a disease that is highly liable to occur due to psychological factors. According to at least one embodiment of the present invention, a therapy (for example, cognitive behavioral therapy) is executed through the information terminal (3) and the information network (10: for example, the Internet) to enable the patient himself to face real psychological factors or deep psychological factors and to resolve psychological factors.

Therefore, a therapeutic effect becomes higher in any one of the psychogenic ED, the organic ED, and the mixed ED.

### BRIEF DESCRIPTION OF THE DRAWINGS

FIG. 1 is a configuration diagram of a system according to at least one embodiment of the present invention.

FIG. 2 is a functional block diagram of a control device used in the at least one embodiment.

FIG. 3 is a functional block diagram of a psychogenic-or-mixed block of FIG. 2.

FIG. 4 is a functional block diagram of a database used in the at least one embodiment.

FIG. 5 is a functional block diagram of a patient-side electronic device used in the at least one embodiment.

FIG. 6 is a functional block diagram of a doctor-side electronic device used in the at least one embodiment.

FIG. 7 is a flow chart for illustrating control in the at least one embodiment.

FIG. 8 is a flow chart of a cognitive behavioral therapy using a chatbot.

FIG. 9 is a flow chart for illustrating an execution procedure for the cognitive behavioral therapy to be performed when a chatbot is not used.

FIG. 10 is a flow chart for transmitting a required exercise, music, video, or other information.

FIG. 11 is a flow chart for illustrating a procedure for a follow-up observation.

FIG. 12 is a flow chart for illustrating the procedure for a follow-up observation.

FIG. 13 is a functional block diagram for illustrating details of a determination block for determining a therapeutic effect through use of a sympathetic nerve marker and a parasympathetic nerve marker.

FIG. 14 is a flow chart of control for determining the therapeutic effect through use of the sympathetic nerve marker and the parasympathetic nerve marker.

FIG. 15 is a functional block diagram for illustrating details of a determination block for determining the therapeutic effect through use of a tensile force which is based on a circumferential expansion of a male genitalia at a time of erection.

FIG. 16 is a flow chart of control for determining the therapeutic effect through use of the tensile force.

FIG. 17 is a functional block diagram for illustrating details of a determination block for determining a psychotherapeutic effect based on reduction of a blood sugar level.

FIG. 18 is a flow chart of control for determining the psychotherapeutic effect through use of the blood sugar level.

FIG. 19 is a functional block diagram for illustrating details of a determination block for determining the therapeutic effect through use of a blood flow velocity.

FIG. 20 is a flow chart of control for determining the therapeutic effect through use of the blood flow velocity.

FIG. 21 is a functional block diagram for illustrating details of a determination block for determining the therapeutic effect through use of a male genitalia surface temperature.

FIG. 22 is a flow chart of control for determining the therapeutic effect through use of the male genitalia surface temperature.

FIG. 23 is an explanatory diagram for illustrating a modification example of an organic block in the at least one embodiment.

FIG. 24 is a functional block diagram of a block for video adjustment illustrated in FIG. 23.

### DESCRIPTION OF THE EMBODIMENTS

Now, at least one embodiment of the present invention is described with reference to the accompanying drawings. In the illustrated at least one embodiment, a case in which a cognitive behavioral therapy is applied as psychotherapy is described.

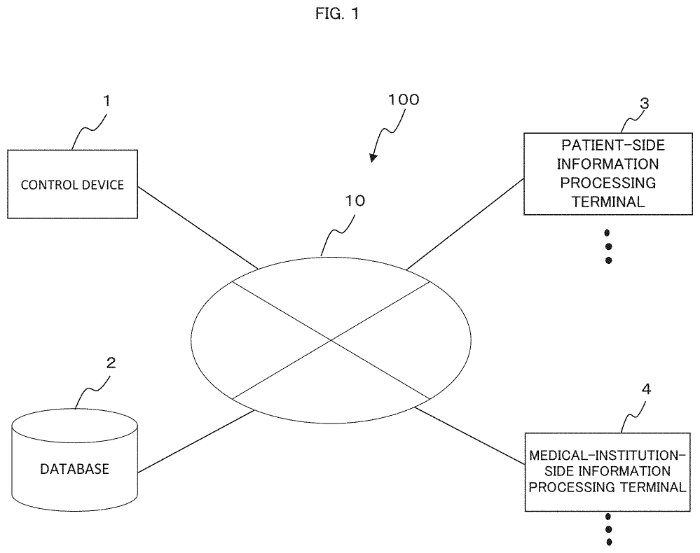

First, with reference to FIG. 1, a configuration of a system for a patient with erectile dysfunction (ED) according to the at least one embodiment of the present invention is described.

In FIG. 1, a system for a patient with ED, which is entirely denoted by reference numeral 100, includes a control device 1 (for example, computer or server), a database 2, an information processing terminal 3 to be used by the patient (patient-side information processing terminal), and an information processing terminal 4 to be used at a medical institution (medical-institution-side information processing terminal), and those components are connected to one another by, for example, an information processing network 10 (for example, local area network or the Internet).

As the patient-side terminal 3 for information processing, it is possible to use an electronic device having an information processing function and a communication function, for example, a smartphone or a personal computer (PC or computer). As the medical-institution-side information processing terminal 4, it is possible to use an electronic device (for example, PC) having an information processing function and a communication function, and to use a smartphone as well.

The database 2 stores information on the patient (for example, diagnosis-and-treatment history information associated with an ID of the patient) and information relating to an erectile dysfunction treatment (for example, treatment method, information relating to a PDE5 inhibitor including a medicine-taking history, and information relating to the cognitive behavioral therapy). Diagnoses and treatments in the diagnosis-and-treatment history include diagnoses and treatments undergone through the information terminal and diagnoses and treatments undergone at the medical institution.

Although not shown in FIG. 1, a plurality of patient-side information processing terminals 3 and a plurality of medical-institution-side information processing terminals 4 can also be connected to the network 10.

In addition, the database 2 can also be directly connected to the control device 1 without being connected to the network 10.

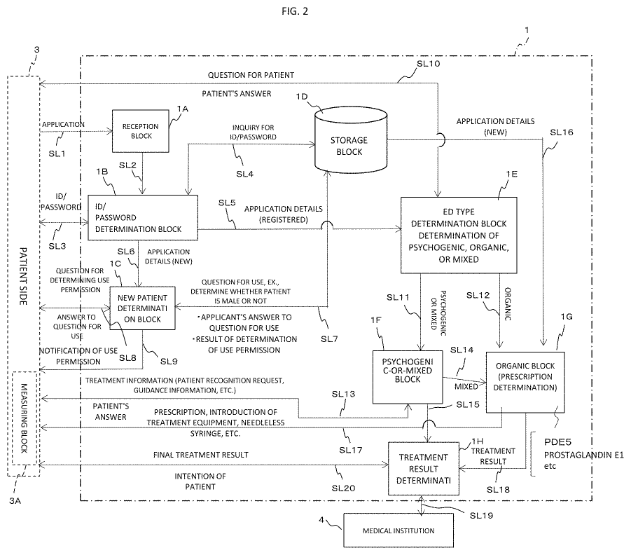

In FIG. 2, which is a functional block diagram of the control device 1, the control device 1 includes a reception block 1A, an ID/password determination block 1B, a new patient determination block 1C, a storage block 1D, an ED type determination block 1E, a psychogenic-or-mixed block 1F, an organic block 1G, and a treatment result determination block 1H, and those functional blocks are connected to one another through signal lines SL.

The reception block 1A has a function of receiving an application for undergoing a treatment of erectile dysfunction (ED) from the patient (patient-side information processing terminal 3) through a signal line SL1, and transmitting application details of the patient to the ID/password determination block 1B through a signal line SL2.

The ID/password determination block 1B has a function of receiving information relating to the application (details) of the patient from the reception block 1A, and transmitting a request for an ID and a password to the patient (patient-side information processing terminal 3) through a signal line SL3 (bidirectional signal line), and also has a function of acquiring, through the signal line SL3, the ID and password transmitted from the patient based on the request.

In addition, the ID/password determination block 1B has a function of inquiring the storage block 1D through a signal line SL4 (bidirectional signal line) for the ID and password acquired from the patient (patient-side information processing terminal 3), and then determining whether the patient is a patient for which an ID and password have already been registered or a new patient for which an ID and password have not been registered.

When the patient is determined to be a registered patient as a result of the determination performed by the ID/password determination block 1B, the ID/password determination block 1B transmits the application details (including ID and password information) received from the patient (patient-side information processing terminal 3) to the ED type determination block 1E through a signal line SL5. Meanwhile, when the patient is a new patient, the ID/password determination block 1B transmits the application details of the new patient to the new patient determination block 1C through a signal line SL6.

In FIG. 2, the new patient determination block 1C has a function of acquiring, when the application details of the new patient are received from the ID/password determination block 1B, a required question (question for use of the system 100 according to the illustrated at least one embodiment) from the storage block 1D through a signal line SL7 (bidirectional signal line), and transmitting the acquired question to the new patient (patient-side information processing terminal 3) through a signal line SL8 (bidirectional signal line). In this case, the acquired question is, for example, a question for determining whether or not the new patient is a male. This is because the use of the system 100 is limited to males.

In addition, the new patient determination block 1C has a function of acquiring the answer from the new patient through the signal line SL8, and determining whether or not the new patient can use the system 100 (for example, whether the new patient is not so-called “prank” or “female impersonating an ED patient”). The determination result obtained by the new patient determination block 1C is returned to the new patient through a signal line SL9. The determination result and the answer from the patient are transmitted to the storage block 1D through the signal line SL7, and stored in the storage block 1D. When the answer from the new patient clarifies that, for example, the new patient is not a female but a male, the new patient determination block 1C determines that the system 100 “can be used,” and replies to the new patient that the system 100 “can be used.” Although not clearly shown, an ID and a password are issued to the new patient at that time.

Meanwhile, when the answer from the new patient has clarified that, for example, “impersonation” is performed by a female, the new patient determination block 1C determines that the system 100 “cannot be used,” and replies to the new patient that the system 100 “cannot be used.”

The question for the use of the system 100 is not only stored in the storage block 1D, but can also be stored in the new patient determination block 1C.

The storage block 1D records the ID and password of the registered patient, other information relating to the patient, the question for use of the system 100 to be asked in response to the application of a new patient, and other such information.

The information stored in the storage block 1D is used by various blocks including the ID/password determination block 1B and the new patient determination block 1C, and is updated by the various blocks.

In FIG. 2, the ED type determination block 1E has a function of exchanging information relating to a symptom with the patient side (patient-side information processing terminal 3), to thereby determine what type of ED (which one of psychogenic ED, organic ED, and mixed ED) the ED of the patient has.

The ED type determination block 1E has a function of transmitting, when the application details of the patient are received through the ID/password determination block 1B, a question about the current symptom to the patient through a signal line SL10 (bidirectional signal line). The ED type determination block 1E has a function of then acquiring the patient's answer to the question through the signal line SL10.

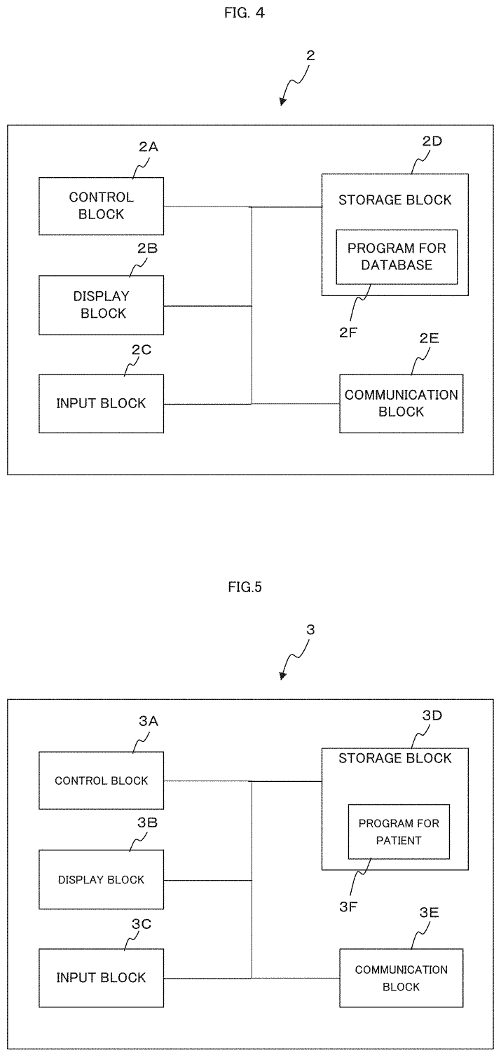

Although not clearly shown, the ED type determination block 1E is also connected to the database 2, which is described later with reference to FIG. 4, and is allowed to refer to the information (for example, diagnosis-and-treatment history information) on the patient and information relating to the ED treatment (for example, treatment method, information relating to the PDE5 inhibitor, and information relating to psychotherapy including the cognitive behavioral therapy), which are stored in the database 2.

The ED type determination block 1E has a function of then determining whether the ED of the patient is the psychogenic ED, the organic ED, or the mixed ED being a mixture of the psychogenic ED and the organic ED based on: details of the patient's answer to the question; and the patient information including the diagnosis-and-treatment history and the information relating to the ED treatment, which are stored in the database 2.

When the patient is determined to have psychogenic ED or mixed ED as a result of the determination performed by the ED type determination block 1E, the ED type determination block 1E transmits the determination result to the psychogenic-or-mixed block 1F through a signal line SL11. Meanwhile, when the patient has organic ED, the ED type determination block 1E transmits the determination result to the organic block 1G through a signal line SL12.

In FIG. 2, the psychogenic-or-mixed block 1F has a function of executing a therapy of psychogenic ED or mixed ED when the determination result (including a basis of the determination) is acquired from the ED type determination block 1E. The psychogenic-or-mixed block 1F also has a function of executing a follow-up observation. The psychogenic-or-mixed block 1F executes merely a part of the follow-up observation, and the treatment result determination block 1H executes determination of a treatment result.

In a case of a treatment, the psychogenic-or-mixed block 1F transmits treatment information (for example, guidance information and patient recognition request in the cognitive behavioral therapy) to the patient-side information processing terminal 3 through a signal line SL13 (bidirectional signal line), and acquires the patient's answer or response to the treatment information. After the exchange of such information and the like is executed (for example, a plurality of times), the treatment of the psychogenic ED or the mixed ED including the follow-up observation is executed.

Although not clearly shown, the psychogenic-or-mixed block 1F is connected to the database 2, and is allowed to use the information (for example, diagnosis-and-treatment history information) on the patient and the information relating to the ED treatment (for example, treatment method, information relating to the PDE5 inhibitor, and information relating to psychotherapy including the cognitive behavioral therapy), which are recorded in the database 2.

In addition, the psychogenic-or-mixed block 1F has a function of transmitting prescriptions for a PDE5 inhibitor and a depression drug, information relating to a needleless syringe and other instruments, and information required for obtaining those (information required for obtaining prescriptions) to the patient through the signal line SL13.

In addition, the information relating to the psychogenic ED, which has been acquired from the patient by the psychogenic-or-mixed block 1F, is transmitted to the organic block 1G through a signal line SL14, and is utilized for executing various functions of the organic block 1G.

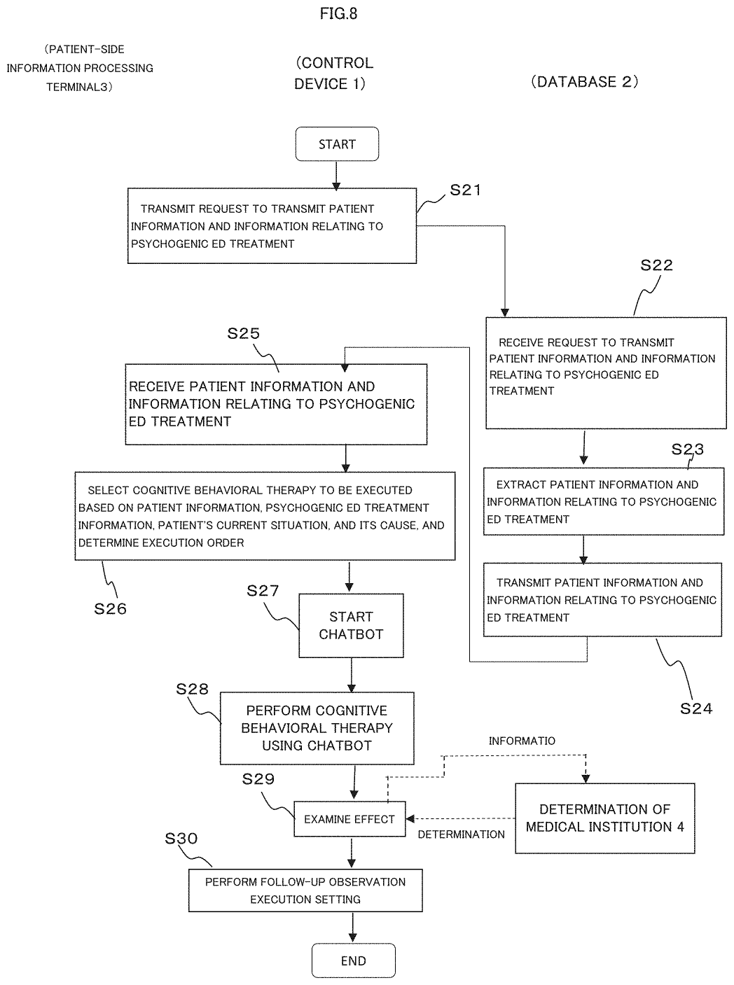

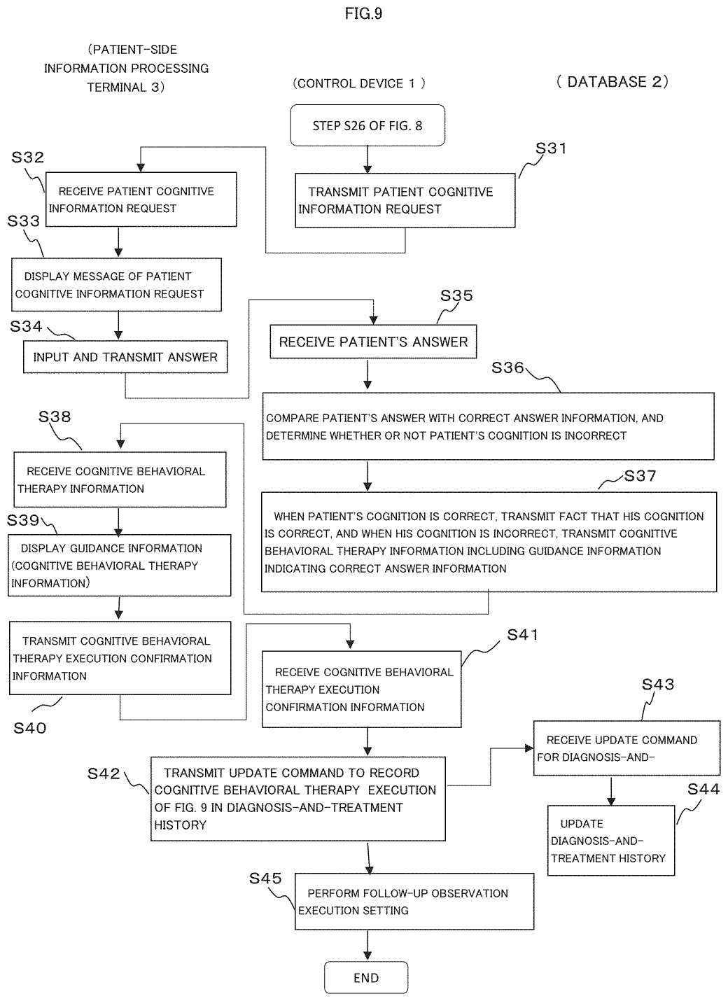

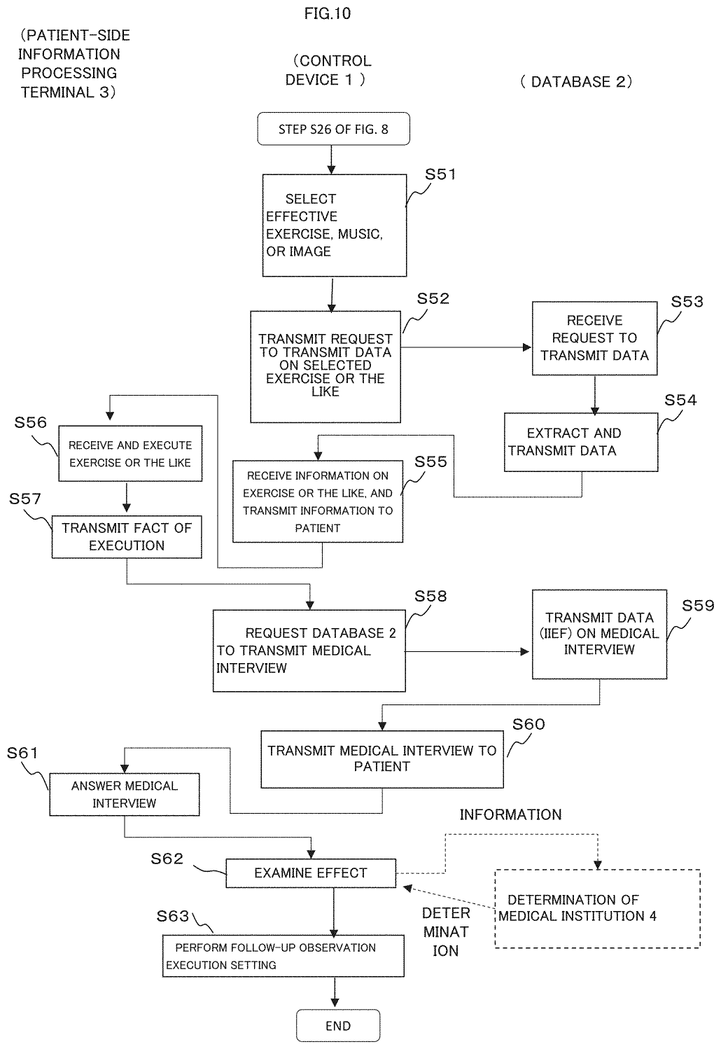

A procedure to be performed by the psychogenic-or-mixed block 1F (procedure relating to the ED treatment) is described later with reference to flow charts of FIG. 8 and FIG. 9.

The treatment result obtained by the psychogenic-or-mixed block 1F is transmitted to the treatment result determination block 1H through a signal line SL15.

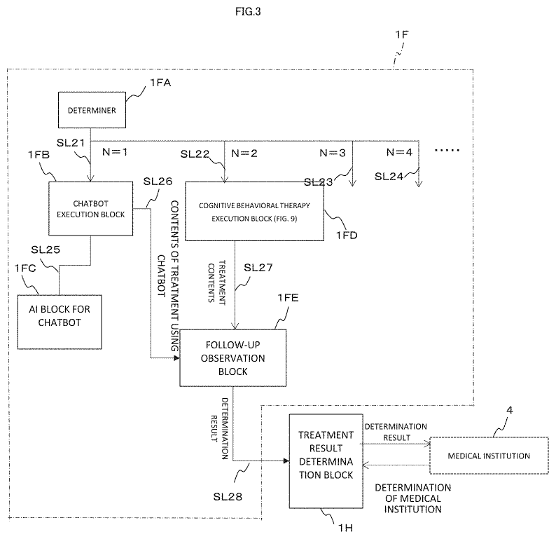

Details of the psychogenic-or-mixed block 1F are described later with reference to FIG. 3.

The organic block 1G has a function of executing a therapy for an organic ED patient when the determination result (including a basis of the determination) that “the patient has organic ED” is acquired from the ED type determination block 1E. The organic block 1G also has a function of executing a follow-up observation. In this case, the organic block 1G executes merely a part of the follow-up observation, and the treatment result determination block 1H executes determination of a treatment result.

In a case of a treatment, the organic block 1G acquires the treatment information including the patient's past prescription from the storage block 1D through a signal line SL16, and determines how to deal with the ED (including use of a PDE5 inhibitor, a prostaglandin E1 preparation, or another drug) through use of the information (for example, diagnosis-and-treatment history information) on the patient and the information relating to the ED treatment, which are stored in the database 2, as well. In the case of the treatment, the organic block 1G transmits the treatment information (including the prescription and an inquiry of treatment equipment) to the patient-side information processing terminal 3 through a signal line SL17.

The treatment result obtained by the organic block 1G is transmitted to the treatment result determination block 1H through a signal line SL18.

In FIG. 2, the treatment result determination block 1H has a function of transmitting, when the treatment result is obtained from the psychogenic-or-mixed block 1F or the organic block 1G, information relating to the treatment result to a medical institution 4 through a signal line SL19 (bidirectional signal line). The treatment result determination block 1H also has a function of receiving a verification result obtained by the medical institution 4 with respect to the transmitted treatment result.

The treatment result determination block 1H has a function of then determining whether or not the treatment result is possible in consideration of the verification result obtained by the medical institution 4. The treatment result determination block 1H may modify the treatment result obtained by the psychogenic-or-mixed block 1F based on the verification performed by the medical institution 4.

However, it is possible to omit the verification performed by the medical institution 4.

In the illustrated at least one embodiment, reference numeral “4” denotes the “medical-institution-side information processing terminal,” but may denote the “medical institution” itself.

The treatment result determination block 1H has a function of transmitting the treatment result to the patient-side information processing terminal 3 through a signal line SL20.

Examples of the treatment result (treatment result subjected to the verification by the medical institution 4) include the description “A therapeutic effect has been confirmed with the most recently executed treatment (for example, cognitive behavioral therapy), and the treatment can be ended at the current time point. In six months from now, the patient's symptoms will be examined again.” and the description “A therapeutic effect has not been confirmed with the most recent treatment. Therefore, the treatment will be continuously executed by changing the treatment contents.”

When the treatment result determination block 1H performs the determination by omitting the verification performed by the medical institution 4, it is preferred that a doctor check the information of the treatment result determination block 1H.

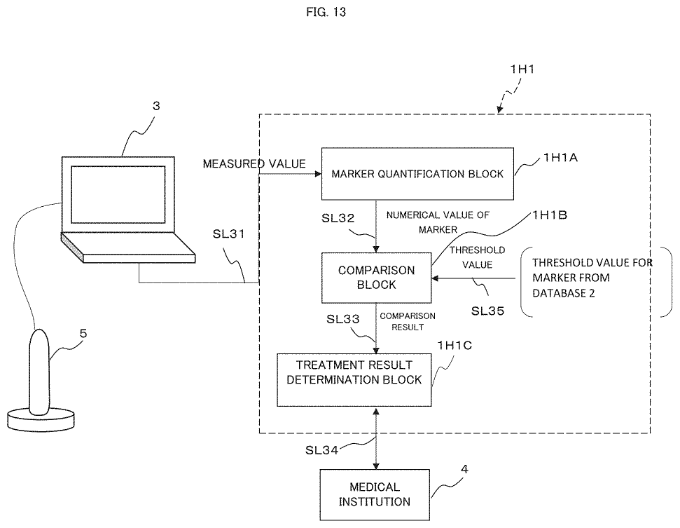

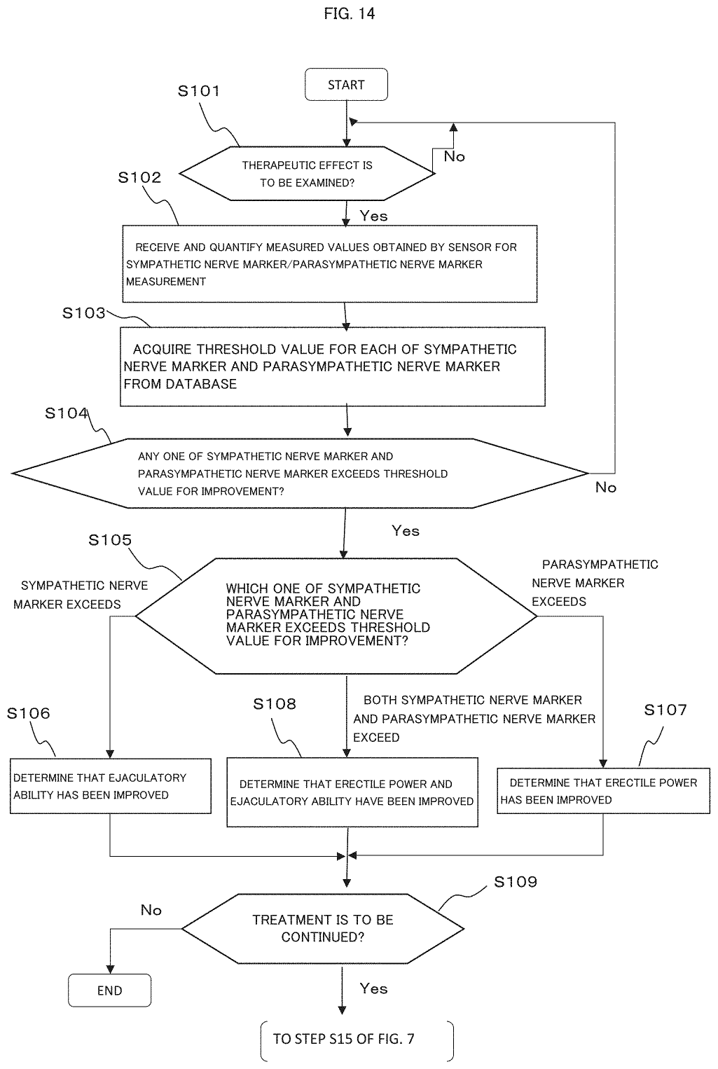

As described later with reference to FIG. 13 to FIG. 22, the patient side 3 (patient-side information processing terminal 3) can be provided with a measuring device for physically verifying a situation of the patient, for example, a measuring block 3A in which a sympathetic nerve/parasympathetic nerve marker sensor, a blood sugar level measuring sensor, a blood flow velocity measuring sensor, and other measuring devices for physically verifying the therapeutic effect are arranged.

In FIG. 3 for illustrating the psychogenic-or-mixed block 1F in detail, the psychogenic-or-mixed block 1F includes a determiner 1FA, a chatbot execution block 1FB, an AI block 1FC for a chatbot, a cognitive behavioral therapy execution block 1FD for executing a cognitive behavioral therapy, and a follow-up observation block 1FE.

The determiner 1FA has a function of receiving the determination result of “having psychogenic ED or mixed ED” from the ED type determination block 1E of the control device 1 illustrated in FIG. 2, and determining the treatment contents and other information to be used in the psychogenic-or-mixed block 1F. The treatment contents are determined through use of the information (for example, diagnosis-and-treatment history information) on the patient and the information relating to the ED treatment (for example, treatment method, information relating to the PDE5 inhibitor, and information relating to psychotherapy including the cognitive behavioral therapy), which are stored in the database 2.

In the illustrated at least one embodiment, the determiner 1FA determines a plurality of treatment contents and an order in which the treatment contents are performed. That is, in the case of the treatment, a cognitive behavioral therapy using a chatbot (conversation robot automated by artificial intelligence (AI)) is executed first (N=1), and a cognitive behavioral therapy (using no chatbot, as illustrated in FIG. 9) is executed second (N=2). After that, a third treatment and a fourth treatment (treatments of N=3 and N=4, for example, other therapies different from the cognitive behavioral therapies) are executed as the requirement arises depending on the course after the first treatment and the second treatment (treatments of N=1 and N=2).

An execution instruction for the first treatment issued from the determiner 1FA, for example, an execution instruction for the cognitive behavioral therapy (N=1) using a chatbot, is transmitted to the chatbot execution block 1FB through a signal line SL21. An execution instruction for the cognitive behavioral therapy (N=2) being the second treatment is transmitted to the cognitive behavioral therapy execution block 1FD through a signal line SL22.

In the same manner, execution instructions for the other therapies of N=3 and N=4 issued from the determiner 1FA are transmitted to execution blocks (not shown) through signal lines SL23 and SL24, respectively.

In FIG. 3, the chatbot execution block 1FB and the AI block 1FC for a chatbot have a function of executing the cognitive behavioral therapy using a chatbot in response to an instruction issued from the determiner 1FA. The chatbot execution block 1FB and the AI block 1FC for a chatbot are connected to each other by a signal line SL25. A treatment using a chatbot is described later with reference to FIG. 8.

The treatment contents of the cognitive behavioral therapy using a chatbot are transmitted from the chatbot execution block 1FB (one by one or periodically) to the follow-up observation block 1FE through a signal line SL26.

The cognitive behavioral therapy execution block 1FD has a function of executing another cognitive behavioral therapy using no chatbot (for example, cognitive behavioral therapy illustrated in FIG. 9) in response to an instruction issued from the determiner 1FA. The treatment performed by the cognitive behavioral therapy execution block 1FD is described later with reference to the flow chart of FIG. 9.

The contents of the cognitive behavioral therapy performed by the cognitive behavioral therapy execution block 1FD are transmitted (one by one or periodically) to the follow-up observation block 1FE through a signal line SL27.

In FIG. 3, the follow-up observation block 1FE has a function of acquiring the treatment contents from the chatbot execution block 1FB, and determining whether or not the treatment using the chatbot including the follow-up observation has been completed.

In the same manner, the follow-up observation block 1FE has a function of acquiring the treatment contents from the cognitive behavioral therapy execution block 1FD, and determining whether or not the treatment including the follow-up observation has been completed.

The determination result obtained by the follow-up observation block 1FE is transmitted to the treatment result determination block 1H, which is also illustrated in FIG. 2, through a signal line SL28.

The treatment result determination block 1H acquires the determination result (treatment result) from the follow-up observation block 1FE, and finally determines the determination result (treatment result) in response to the verification of the treatment result by the medical institution 4.

The functions of the treatment result determination block 1H including a relationship with the medical institution 4 and information exchange with the patient (patient-side information processing terminal 3) are as described above with reference to FIG. 2.

In FIG. 4, the database 2 has a function of storing the information on the patient and the information relating to the erectile dysfunction treatment, and includes a control block 2A, a display block 2B, an input block 2C, a storage block 2D, and a communication block 2E.

The control block 2A has a function of executing control for performing, for example, information processing in the database 2. The display block 2B has a function of displaying information to a user of the database 2 (for example, doctor or person in charge at the medical institution). The input block 2C has a function of receiving information input through, for example, a keyboard.

The storage block 2D is formed of an existing device for storage, and stores a program 2F for a database. The storage block 2D also stores the information on the patient (for example, diagnosis-and-treatment history information associated with a patient ID) and the information relating to the erectile dysfunction treatment (for example, the treatment method, the information relating to the PDE5 inhibitor, the information relating to the cognitive behavioral therapy, and information relating to prescription). The communication block 2E has a function of executing wired or wireless communication to connect the database 2 to the network 10.

The information stored in the database 2 is transmitted by, for example, the control device 1 as the requirement arises. In addition, the information is updated based on the information received from the control device 1, the patient-side information processing terminal 3, and the medical-institution-side information processing terminal 4.

The control device 1 and the database 2 can also be formed as a single computer.

In this case, the term “patient” in the illustrated at least one embodiment means an individual who suffers from erectile dysfunction and is willing to treat the erectile dysfunction, and is not limited to one who is undergoing the treatment under guidance of a doctor or another person concerned at a medical institution.

In addition, the patient-side information processing terminal 3 is not particularly limited as long as the patient-side information processing terminal 3 is an electronic device having an information processing function and a communication function. However, a portable information terminal, for example, a so-called smartphone, is preferred in consideration of its recent spread.

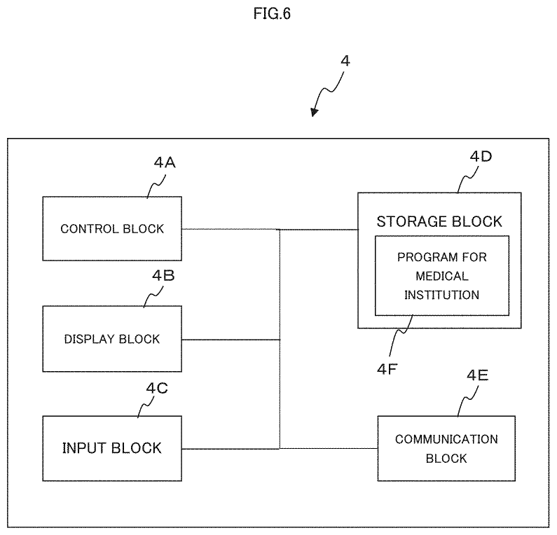

In FIG. 5, the patient-side information processing terminal 3 includes a control block 3A, a display block 3B, an input block 3C, a storage block 3D, and a communication block 3E.

The control block 3A has a function of executing control for performing, for example, information processing in the patient-side information processing terminal 3. The display block 3B has a function of displaying information to a user (patient) of the patient-side information processing terminal 3. The input block 3C has a function of receiving information input by the user (patient).

The storage block 3D is formed of an existing device for storage, and stores a program for a patient, which is denoted by reference symbol 3F, for the patient-side information processing terminal 3. The communication block 3E has a function of connecting the patient-side information processing terminal 3 to the network 10 by wired or wireless communication.

Although not shown in FIG. 5, the patient-side information processing terminal 3 can be provided with a measuring device for physically verifying a situation of the patient, as illustrated in FIG. 2.

In FIG. 6, the medical-institution-side information processing terminal 4 includes a control block 4A, a display block 4B, an input block 4C, a storage block 4D, and a communication block 4E.

The control block 4A has a function of executing control for performing, for example, information processing in the medical-institution-side information processing terminal 4. The display block 4B has a function of displaying information to a user of the medical-institution-side information processing terminal 4 (for example, doctor or person in charge at the medical institution). The input block 4C has a function of receiving information input by, for example, the doctor or the person in charge at the medical institution.

The storage block 4D is formed of an existing device for storage, and stores a program for a medical institution, which is denoted by reference symbol 4F, for the medical-institution-side information processing terminal 4. The communication block 4E has a function of connecting the medical-institution-side information processing terminal 4 to the network 10 by wired or wireless communication.

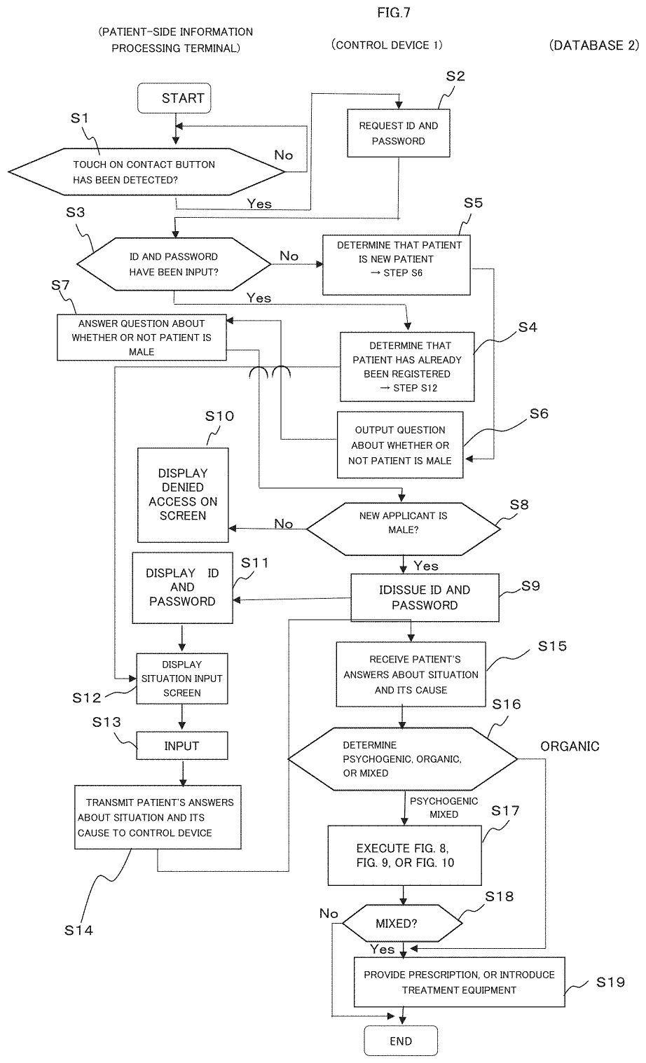

Next, an execution procedure of the system 100 for an erectile dysfunction (ED) patient according to the at least one embodiment is described with reference to FIG. 7 to FIG. 12.

The patient-side information processing terminal 3 is exemplified by a smartphone, and the medical-institution-side information processing terminal 4 is exemplified by a so-called “laptop” PC. In this case, a case in which a single patient-side information processing terminal 3 and a single medical-institution-side information processing terminal 4 are used is described as an example. However, the system 100 may include a plurality of patient-side information processing terminals 3 and a plurality of medical-institution-side information processing terminals 4.

In the illustrated at least one embodiment, as illustrated in FIG. 7, when the patient operates the patient-side information processing terminal 3, information indicating a current situation of the erectile dysfunction of the patient is input, and a cognitive behavioral therapy is executed.

In FIG. 7, at a time of performing an ED treatment through use of the system 100, in Step S1, when the patient considers that a problem relating to ED has occurred, the patient touches a contact button (not shown) on the display block 3B of the patient-side information processing terminal 3, and the patient-side information processing terminal 3 detects the touch.

When the touch on the contact button is detected in Step S1 (“Yes” in Step S1), that fact is transmitted from the communication block 3E of the patient-side information processing terminal 3 to the control device 1, and the process advances to Step S2. When the touch on the contact button is not detected, the process returns to Step S1 (“No” loop in Step S1).

In Step S2 (when the touch on the contact button is detected in Step S1), the reception block 1A of the control device 1 receives information on the detection (information indicating that the touch on the contact button has been detected). Having received such a signal means that an application for use of the system 100 has been performed. In response to the fact that the application for use of the system 100 has been performed, the ID/password determination block 1B requests the patient-side information processing terminal 3 for the ID and password.

In Step S3, the input block 3C of the patient-side information processing terminal 3 determines whether or not the ID and password of the patient have been input.

When the ID and password of the patient are input in Step S3 (“Yes” in Step S3), the input ID and password are transmitted to the control device 1 through the communication block 3E, and the process advances to Step S4. In Step S4, when the ID and password input by the patient are genuine, the ID/password determination block 1B of the control device 1 determines that the patient is an already registered patient, and the process advances to Step S12 in order to start the treatment.

Meanwhile, when the ID and password of the patient are not input in Step S3 (“No” in Step S3), the process advances to Step S5. In Step S5, the control device 1 determines that the patient is a new patient (unregistered patient), and the process advances to Step S6.

In Step S6, the new patient determination block 1C of the control device 1 transmits a question about “whether or not the new patient is a male” to the patient-side information processing terminal 3 in order to determine whether or not the new patient can use the system. This is because it is a male that is harassed with ED, and access from a female is liable to be “impersonation.”

In Step S7, the new patient answers the question transmitted in Step S6 through the patient-side information processing terminal 3, and transmits the answer to the control device 1.

In Step S8, the new patient determination block 1C of the control device 1 determines whether or not the new patient is a male based on the answer from the new patient. When it is determined in Step S8 that the new patient is a male (“Yes” in Step S8), the process advances to Step S9, and when it is determined in Step S8 that the new patient is not a male (“No” in Step S8), the process advances to Step S10.

In Step S9 (when it is determined that the new patient is a male), the control device 1 issues an ID and a password to the new patient. The control device 1 displays the ID and password issued to the new patient on the display block 3B of the patient-side information processing terminal 3 (Step S11), to thereby allow the new patient to whom the ID and password have been assigned to use the system 100, and the process advances to Step S12.

Meanwhile, in Step S10 (when it is determined that the new patient is not a male), the new patient determination block 1C of the control device 1 transmits an answer of denied access to the system 100 to the patient-side information processing terminal 3 of the new patient, and the answer of “denied access” is displayed on the display block 3B of the patient-side information processing terminal 3.

Both the registered patient and the patient to whom the ID and password are newly assigned can start to use the system 100 when the process advances to Step S12.

In Step S12 of FIG. 7, the ED type determination block 1E of the control device 1 transmits a question for asking what situation the patient is currently in to the patient-side information processing terminal 3, and the question is displayed on a situation input screen of the display block 3B on the patient side.

The question is, for example, “What happened? Please select one that is close to your current situation. 1: Recently, I have been continuously unable to get an erection. 2: Recently, I have been often unable to get an erection. 3: Recently, I get an insufficient erection that does not last.” The patient selects an applicable one of the three situations in the question. In this case, it is also possible to display an option indicating a situation other than the above-mentioned three situations on the display block 3B. It is also possible to allow the patient to freely input his own situation instead of selecting the option.

In this case, as the question for asking the current situation of the patient, for example, it is preferred to adopt the International Index of Erectile Function (IIEF).

The patient inputs an answer (for example, option) indicating the current situation he is considering to the input block 3C of the patient-side information processing terminal 3 (Step S13). In this case, a case in which the patient's answer is “2: I have been often unable to get an erection.” is described as an example. In Step S13, the patient can be asked to answer the cause of the current situation by himself.

Then, in Step S14, the patient's answer is transmitted from the communication block 3E to the control device 1 together with the patient ID that identifies the patient.

In this case, in place of Step S12 to Step S14, after the patient has applied for the use of the system 100 and the ID and password have been confirmed, the information required for the patient to input and display the situation and other information can be transmitted from the control device 1 to the patient-side information processing terminal 3.

In Step S15 of FIG. 7, the ED type determination block 1E of the control device 1 receives the patient ID and the patient's answer (answer indicating the current situation). Then, the process advances to Step S16, and the ED type determination block 1E identifies (determines) whether the type of the patient's ED is “psychogenic ED,” “organic ED,” or “mixed ED” based on the answer (information) indicating the current situation of the patient and/or through use of the information (for example, the diagnosis-and-treatment history and other kinds of patient information, information relating to the ED treatment, information relating to the PDE5 inhibitor, and information relating to the cognitive behavioral therapy) stored in the database 2 (not shown) as well.

When the determination result of Step S16 is psychogenic ED or mixed ED, the process advances to Step S17, and when the determination result of Step S16 is organic ED, the process advances to Step S19.

In Step S17, the psychogenic-or-mixed block 1F of the control device 1 executes the treatment of the psychogenic ED. In the illustrated at least one embodiment, for example, the cognitive behavioral therapy using a chatbot (execution procedure therefor is illustrated in FIG. 8), the cognitive behavioral therapy using no chatbot (execution procedure therefor is illustrated in FIG. 9), and a therapy using an exercise, music, video, or other content (execution procedure therefor is illustrated in FIG. 10) are executed as the treatment provided for the psychogenic ED.

In Step S18, it is determined whether or not the patient has mixed ED. This is because the treatment of Step S17 is effective for a part corresponding to the psychogenic ED in the mixed ED but the treatment provided for the organic ED is effective for a part corresponding to the organic ED.

In the case of mixed ED in Step S18 (“Yes” in Step S18), the process advances to Step S19. When the determination result of Step S18 is not mixed ED (“No” in Step S18), the treatment required for the psychogenic ED is already performed in Step S17, and hence the control illustrated in FIG. 7 is ended.

In Step S19, the patient has any one of organic ED and mixed ED. The organic block 1G of the control device 1 executes the treatment provided for the organic ED. As the treatment provided for the organic ED, for example, it is possible to execute the treatment based on a prescription (for, e.g., a PDE5 inhibitor or a prostaglandin E1 preparation) as well as introduction of equipment.

A procedure for a treatment using a cognitive behavioral therapy using a chatbot, which is performed when it is determined in Step S16 of FIG. 7 that the patient has “psychogenic ED,” is illustrated in detail in FIG. 8.

In FIG. 8, in Step S21, the control device 1 transmits, to the database 2 based on the patient ID, a request to transmit the patient information on the patient to the control device 1. At the same time, in Step S21, the control device 1 transmits, to the database 2, a request to transmit the information (for example, treatment method, information relating to the PDE5 inhibitor, and information relating to the cognitive behavioral therapy) relating to a psychogenic erectile dysfunction treatment, which is stored in the database 2, to the control device 1.

In Step S22, the communication block 2E of the database 2 receives such transmission requests from the control device 1. Then, in Step S23, the control block 2A of the database 2 extracts the patient information on the patient and the information relating to the psychogenic erectile dysfunction treatment, which are stored in the storage block 2D, based on the patient ID received together with the patient information transmission request, and in Step S24, the communication block 2E of the database 2 transmits the extracted information to the control device 1.

In Step S25, the control device 1 receives the patient information and the information relating to the psychogenic erectile dysfunction treatment (for example, treatment method, information relating to the PDE5 inhibitor, and information relating to the cognitive behavioral therapy) from the database 2.

In Step S26, the psychogenic-or-mixed block 1F of the control device 1 selects a cognitive behavioral therapy appropriate for (to be executed on) the current patient from a therapy list based on the patient information, the information relating to the psychogenic erectile dysfunction treatment, and information relating to the patient's current physical condition (situation), which have been received. In Step S26, a plurality of treatment methods can be selected, and an order in which the plurality of selected treatment methods are executed can also be determined.

In the illustrated at least one embodiment, it has already been confirmed in Step S16 of FIG. 7 that the patient has psychogenic ED (or mixed ED as well). As described with reference to the configuration diagram of the psychogenic-or-mixed block 1F of FIG. 3, the cognitive behavioral therapy using a chatbot is first executed (N=1), and then the cognitive behavioral therapy using no chatbot, which is illustrated in FIG. 9, is executed (N=2). In addition, therapies other than the cognitive behavioral therapies can be executed as the requirement arises (N=3 and N=4).

In the illustrated at least one embodiment, the information relating to the psychogenic erectile dysfunction treatment is stored in the database 2 together to be utilized by the control device 1 in Step S26. However, the information relating to the psychogenic erectile dysfunction treatment can also be previously stored in the storage block 1D of the control device 1.

In FIG. 8, in Step S27, a conversation (email exchange) with the patient is started by a chatbot, which is a conversation robot automated by artificial intelligence (AI). An environment is maintained so that required information can be smoothly exchanged during a conversation (email exchange) using a chatbot.

In Step S28, the psychogenic-or-mixed block 1F (more specifically, chatbot execution block 1FB and AI block 1FC for a chatbot, which are illustrated in FIG. 3) of the control device 1 executes the cognitive behavioral therapy on the patient. The patient can be aware of, for example, a cognitive distortion and rewrite his cognition while having a conversation (email exchange) using a chatbot. That is, through the conversation (email exchange), the patient's cognition relating to psychological factors is accurately and effectively rewritten by AI, and the patient's cognition relating to ED is directly rewritten, to thereby execute the treatment of the psychogenic ED.

The conversation (email exchange) using a chatbot is also executed during the follow-up observation.

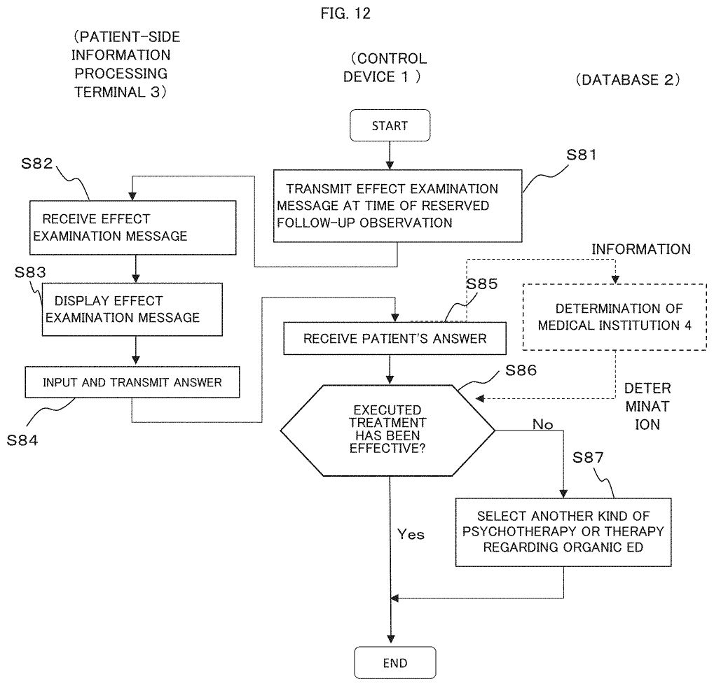

In Step S29, the treatment result determination block 1H of the control device 1 performs an effect examination of the behavioral therapy using a chatbot. At a time of the effect examination, the control device 1 subjects the patient to the effect examination based on a medical interview (described later with reference to FIG. 12) and/or a physical effect examination based on physiological data on the patient (described later with reference to FIG. 13 to FIG. 22).

In addition, in Step S29, information on a result of the effect examination is transmitted to the medical institution 4. The medical institution 4 verifies the result of the effect examination, determines whether the treatment is effective, and then transmits a result of the determination to the treatment result determination block 1H.

In Step S30, the treatment result determination block 1H of the control device 1 performs follow-up observation execution setting when a predetermined therapeutic effect is confirmed in Step S29 in consideration of the result of the determination performed by the medical institution 4.

Although not clearly shown, when a predetermined therapeutic effect cannot be confirmed in Step S29 in consideration of the result of the determination performed by the medical institution 4, the treatment is continued as well as investigation of execution or adoption of another treatment method.

FIG. 9 is a flow chart for illustrating a procedure to be performed when a cognitive behavioral therapy other than the cognitive behavioral therapy using a chatbot is adopted (see Step S26 of FIG. 8).

In FIG. 9, in Step S31, the psychogenic-or-mixed block 1F of the control device 1 (more specifically, cognitive behavioral therapy execution block 1FD illustrated in FIG. 3) transmits, to the patient-side information processing terminal 3, a patient cognitive information request for inquiring the patient's cognition relating to the current physical condition (situation) based on the cognitive behavioral therapy (therapy for directly rewriting the patient's cognition in terms of the ED itself).

In Step S32, the patient-side information processing terminal 3 receives the request for the information relating to the current physical condition (situation) (message: patient cognitive information request for inquiring the patient's cognition), which is transmitted in Step S31. Then, in Step S33, the received patient cognition information request (message) is displayed on the display block 3B.

In this case, the cognitive behavioral therapy is to directly rewrite the patient's cognition in terms of the ED itself, and the message of the patient cognitive information request displayed in Step S33 is a message for inquiring the patient's cognition in terms of having ED.

In the illustrated at least one embodiment, the message of the patient cognitive information request is, for example, “You seem to be suffering from ED due to psychological factors. Do you think that a male with ED lacks value as a male? 1: He lacks value as a male. 2: It cannot be said that he lacks value as a male, but I feel that he lacks value in terms of social role. 3: He does not lack value as a male. His humanity and work ability determine the value of a male.” As such a message, for example, it is preferred to adopt content in light of the International Index of Erectile Function (IIEF).

In Step S34, the patient inputs an answer (option) considered to be correct to the input block 3C of the patient-side information processing terminal 3. An exemplary case in which the patient has selected the answer that the ED patient “lacks value as a male” is described below.

The answer input in Step S34 is transmitted to the control device 1. Then, in Step S35, the control device 1 receives the patient's answer input in Step S34.

In Step S36, the psychogenic-or-mixed block 1F of the control device 1 compares the received patient's answer information with correct answer information (information included in cognitive behavioral therapy information relating to the erectile dysfunction treatment, which has already been acquired from the database 2), and determines whether or not the patient's cognition of the fact itself that he has erectile dysfunction is appropriate (whether or not the patient's cognition is correct).

In FIG. 9, when the patient's cognition is correct in Step S36, in Step S37, the fact that the patient's cognition is correct is transmitted to the patient-side information processing terminal 3. When the patient's cognition is incorrect in Step S36, in Step S37, the cognitive behavioral therapy information including the guidance information indicating the correct answer information is transmitted to the patient-side information processing terminal 3.

In the example described above with reference to FIG. 9, the correct answer to the patient cognitive information for the cognitive behavioral therapy is “3: He does not lack value as a male. His humanity and work ability determine the value of a male.”, and “1: He lacks value as a male.” being the patient's answer (answer described as an example) in Step S36 is incorrect. Therefore, in the above-mentioned example, in Step S37, it is determined that the patient's answer is incorrect, and the guidance information indicating the correct answer information is transmitted. The guidance information is, for example, “Having ED does not mean that he lacks value as a male, and it is his humanity and work ability that determine the value of a male. Please live with confidence as you are.”

Unlike in the above-mentioned example, when the patient answers correctly in Step S36, as the guidance information, for example, the message “Your cognition is correct. Please live with confidence as you are.” is transmitted (Step S37).

In Step S38, the patient-side information processing terminal 3 receives the cognitive behavioral therapy information including the guidance information indicating the correct answer information transmitted in Step S37. In Step S39, the guidance information of the cognitive behavioral therapy information is displayed on the display block 3B of the patient-side information processing terminal 3. Through reception of the guidance information, the patient can recognize that he has misunderstood about erectile dysfunction, to thereby be able to rewrite the patient's cognition relating to erectile dysfunction. Thus, the patient can be expected to recover from the state of psychogenic ED.

After the patient executes the cognitive behavioral therapy (through use of the patient-side information processing terminal 3) in Step S32 to Step S39, in Step S40, the patient-side information processing terminal 3 transmits, to the psychogenic-or-mixed block 1F of the control device 1, cognitive behavioral therapy execution confirmation information indicating that the cognitive behavioral therapy has been executed.

In Step S41, the control device 1 receives the cognitive behavioral therapy execution confirmation information transmitted from the patient side in Step S40. Then, in Step S42, the control device 1 transmits, to the database 2, an update command to record the cognitive behavioral therapies executed up to that time point in the diagnosis-and-treatment history and update the diagnosis-and-treatment history.

In Step S43, the database 2 receives the update command from the control device 1, and in Step S44, the diagnosis-and-treatment history is updated based on the update command.

In Step S41, an examination message can also be transmitted from the control device 1 to the patient-side information processing terminal 3 to obtain an answer thereto from the patient. This is because, after it is confirmed that the cognitive behavioral therapy has been executed on the patient-side information processing terminal 3, it is examined whether or not, owing to the cognitive behavioral therapy, the patient has recognized that he had false cognition and has acquired correct cognition. In that case, the answer from the patient is transmitted from the control device 1 to the database 2, and the diagnosis-and-treatment history is updated.

After Step S42, the control device 1 executes the procedure of Step S45. In Step S45, the follow-up observation block 1FE of the psychogenic-or-mixed block 1F performs the follow-up observation execution setting for setting a timing to execute the follow-up observation.

Next, with reference to FIG. 10, a procedure to be performed when the therapy using an exercise (for example, attention training) in the cognitive behavioral therapy is selected in Step S26 of FIG. 8 is described.

In FIG. 10, in Step S51, the psychogenic-or-mixed block 1F of the control device 1 selects an exercise, music, or video that is therapeutically effective for the patient. At a time of the selection, related information including patient information accumulated in the database 2 and the information relating to the psychogenic erectile dysfunction treatment is taken into consideration (used) as the requirement arises.

In Step S52, the control device 1 requests the database 2 for data on the exercise, music, or video selected in Step 351 (transmission request).

In Step S53, the communication block 2E of the database 2 receives the transmission request from the control device 1. Then, in Step S54, the control block 2A of the database 2 extracts the data on the exercise (or music or video) selected in Step S52 from the storage block 2D, and the communication block 2E of the database 2 transmits the extracted data on exercise (or music or video) to the control device 1.

In Step S55, the psychogenic-or-mixed block 1F of the control device 1 receives the data on the exercise (or music or video) transmitted from the database 2. Then, the psychogenic-or-mixed block 1F transmits the data to the patient-side information processing terminal 3.

In Step S56, the patient-side information processing terminal 3 receives the data on the exercise (or music or video), and the patient executes the exercise (or music or video) through use of the patient-side information processing terminal 3 or a medium on which the exercise (or music or video) can be reproduced.

In Step S57, the patient who has executed the exercise (or music or video) uses the patient-side information processing terminal 3 to transmit, to the control device 1, a report that he has executed the exercise (or music or video).

In Step S58, the control device 1 receives the report that the patient has executed the exercise (or music or video), and requests the database 2 to transmit a medical interview for examining an effect thereof (transmission request).

In Step S59, the database 2 receives the transmission request for the medical interview from the control device 1, extracts data on the medical interview, and transmits the extracted data to the control device 1. In regard to the details of the medical interview, for example, it is preferred to adopt the International Index of Erectile Function (IIEF).

In Step S60, the psychogenic-or-mixed block 1F of the control device 1 transmits the medical interview acquired from the database 2 to the patient-side information processing terminal 3.

In Step S61, the patient examines the details of the medical interview displayed on the display block 3B of the patient-side information processing terminal 3, answers the medical interview through the input block 3C, and transmits the answer to the control device 1.

Examples of the details of the medical interview include “Is there any change in your mental and physical states by executing the exercise (or music or video)? Please select a suitable one. 1: I'm now in a very refreshed and relaxed state. 2: I feel a little better, but I don't think this state will last for a long time. 3: Nothing has changed since before I started to execute the exercise (or music or video).” In this case, it should be noted that this example sentence does not adopt the International Index of Erectile Function (IIEF).

In addition, the answer to the medical interview is not required to be selected from among the options, and it is possible to allow the patient to freely input his own situation as the answer.

In Step S62, the control device 1 receives the patient's answer, and the follow-up observation block 1FE of the psychogenic-or-mixed block 1F examines (determines) (the treatment result determination block 1H can also determine) the effect of the exercise (or music or video) executed by the patient from the content of the answer.

In the illustrated at least one embodiment, when the effect is to be examined by the control device 1, the control device 1 provides the medical institution 4 with information on the exercise (or music or video) executed by the patient, the content of the patient's answer to the medical interview, the patient's treatment history, and other information, and then carries out the effect examination after acquiring the determination of the medical institution 4. However, it is also possible to omit the determination of the medical institution 4, and in that case, the doctor performs the determination after checking the information on the effect examination performed by the treatment result determination block 1H.

In Step S63, the psychogenic-or-mixed block 1F (or treatment result determination block 1H) performs the follow-up observation execution setting when a predetermined therapeutic effect is confirmed in Step S62 in consideration of the result of the determination performed by the medical institution 4 as well.

Although not clearly shown, when a predetermined therapeutic effect cannot be confirmed in Step S62 in consideration of the result of the determination performed by the medical institution 4, the treatment can be continued through use of another exercise (or music or video) as the requirement arises.

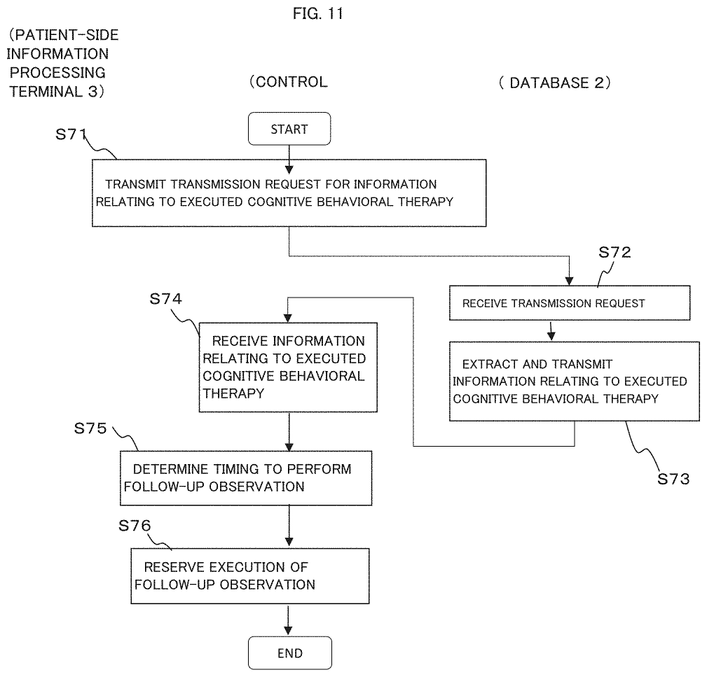

Next, a procedure for the follow-up observation execution setting is described with reference to FIG. 11.

In FIG. 11, in Step S71, the control device 1 requests the database 2 to transmit information relating to the therapy executed by the patient (for example, therapy described with reference to each of FIG. 8, FIG. 9, and FIG. 10) (transmission request).

In Step S72, the communication block 2E of the database 2 receives the transmission request from the control device 1, and then in Step S73, the control block 2A of the database 2 extracts the information relating to the therapy executed from the storage block 2D, and the communication block 2E of the database 2 transmits the information to the control device 1.

In Step S74, the psychogenic-or-mixed block 1F of the control device 1 receives the information relating to the therapy executed by the patient.

In Step S75, the psychogenic-or-mixed block 1F of the control device 1 determines a timing to perform the follow-up observation as, for example, one day after the execution of the cognitive behavioral therapy.

In Step S76, it is reserved to execute the follow-up observation one day after the execution of the cognitive behavioral therapy.

In Step S74, the information (information relating to the therapy executed by the patient) acquired from the database 2 by the control device 1 includes whether or not the follow-up observation is required. Therefore, unlike in the illustrated example, when the cognitive behavioral therapy executed by the patient does not require the follow-up observation, the flow chart of FIG. 8 is ended without the execution of Step S76. The cognitive behavioral therapy that does not require the follow-up observation can be executed as a periodically executed process.

Next, a procedure for follow-up observation execution processing is described with reference to FIG. 12.

In FIG. 12, in Step S81, the psychogenic-or-mixed block 1F of the control device 1 transmits an effect examination message for examining whether or not the situation of the patient has been improved (for example, the International Index of Erectile Function (IIEF) or a message created with reference thereto) to the patient-side information processing terminal 3 at a predetermined timing to execute the follow-up observation set in Step S45 of FIG. 9 and reserved in Step S76 of FIG. 11 (when a predetermined time period has elapsed since the execution of the treatment method).

In Step S82, the patient-side information processing terminal 3 receives the effect examination message, and in Step S83, displays the received effect examination message on the display block 3B. When the patient has undergone the treatment of the psychogenic ED, the effect examination message is, for example, “Have anxieties in daily life and the mental uneasiness due to a worry been reduced?1: Yes. They have been reduced certainly (or even slightly). 2: No. Nothing has changed since before the treatment.”

In Step S84, the patient inputs an applicable answer (option) into the input block 3C of the patient-side information processing terminal 3.

The patient's answer input in Step S84 is transmitted to the control device 1.

In FIG. 12, in Step S85, the patient response input in Step S84 is received by the control device 1.

In Step S86, the psychogenic-or-mixed block 1F (or treatment result determination block 1H) determines whether or not the patient's answer input in Step S84 is “1: Yes” (namely, effective).

In this case, in the illustrated at least one embodiment, in the stage of Step S85, the medical institution 4 is provided with information on the cognitive behavioral therapy executed by the patient, the content of the patient's answer to the effect examination message, the patient's treatment history, and other information, and after the determination of the medical institution 4 as to whether or not the executed cognitive behavioral therapy has been effective is acquired, the determination of Step S86 is executed. However, it is also possible to omit the determination of the medical institution 4, and in that case, for example, the doctor can perform the determination after checking the information on the effect examination performed by the treatment result determination block 1H.

When the patient's answer is “1: Yes” and it can be determined that the cognitive behavioral therapy has been effective in combination with the determination of the medical institution 4 (“Yes” in Step S86), it is determined that the executed cognitive behavioral therapy has produced a certain effect of reducing the patient's mental uneasiness, and the follow-up observation is ended.

Meanwhile, when the patient's answer is “2: No” and it cannot be determined that the cognitive behavioral therapy has been effective in combination with the determination of the medical institution 4 (“No” in Step S86), no effect of reducing the mental anxiety has been recognized, and hence in Step S87, another cognitive behavioral therapy or a therapy regarding organic ED is selected.

Although not clearly shown, the effect is examined at an appropriate timing after the execution of the selected cognitive behavioral therapy, and when the situation (for example, physical condition) of the patient has not yet been improved, it becomes possible to execute an appropriate cognitive behavioral therapy for further improvement.

In addition, the follow-up observation execution processing can be repeated at predetermined intervals until the physical condition is improved.

In the illustrated at least one embodiment, in a cognitive behavioral therapy performed when psychological factors are considered to be deep psychological factors, the message of the patient cognitive information request transmitted from the control device 1 to the patient-side information processing terminal 3 (message transmitted in Step S31 of FIG. 9) can be, for example, “You seem to have a potential worry about a past event rather than daily life. Do you think it difficult to solve? 1: It is difficult for me to have mental control. 2: Time may solve it, but it will take a long time. 3: I want to take time to solve it without rushing even if it is slow. (I want to receive specialized consulting as required).”

In this case, the correct answer to the patient cognitive information is “3: I want to take time to solve it without rushing even if it is slow.”, and the guidance information indicating the correct answer information to be transmitted from the control device 1 to the patient-side information processing terminal 3 is, for example, “Please take your time to solve the problem without rushing even if it is slow. It may be effective to receive specialized consulting. As you progress toward a solution, you will gradually be released from your mental uneasiness, and your medical condition will improve.”

The effect examination message can be, for example, “Will it be possible to solve your worry relating to a past event? 1: Yes. It may take some time, but I am confident. 2: No. Nothing has changed since before the treatment.”

In FIG. 12, presence or absence of the effect of the ED treatment is determined based on the patient's self-report. Meanwhile, it is possible to determine presence or absence of the effect of the treatment by physical measurement.

FIG. 13 to FIG. 22 are illustrations of modes (modification examples) in which it is determined whether or not the treatment is effective by measuring physical parameters instead of the patient's self-report.

First, with reference to FIG. 13 and FIG. 14, a mode in which the therapeutic effect is determined by a sympathetic nerve marker/parasympathetic nerve marker is described.

The sympathetic nerve marker and the parasympathetic nerve marker are electrical pulses of muscles for moving the heart, and can be measured by attaching sensors to the ears and fingers. It is known that an erectile power is improved when a numerical value of the parasympathetic nerve marker increases, and an ejaculatory ability is improved when a numerical value of the sympathetic nerve marker increases.

Therefore, when, for example, the numerical values of the parasympathetic nerve marker and sympathetic nerve marker of the patient both increase, it can be estimated that the erectile power and the ejaculatory ability have been improved by the effect of the ED treatment.

FIG. 13 is an illustration of details of an effect determination block 1H1 using at least a sympathetic nerve marker, for determining a therapeutic effect by the sympathetic nerve marker/parasympathetic nerve marker (sympathetic nerve marker and/or parasympathetic nerve marker). The effect determination block 1H1 is responsible for a part of the function of the treatment result determination block 1H illustrated in FIG. 2.

In FIG. 13, the effect determination block 1H1 using at least a sympathetic nerve marker includes a marker quantification block 1H1A, a comparison block 1H1B, and a treatment result determination block 1H1C.

A sympathetic nerve marker/parasympathetic nerve marker measurement sensor 5 is connected to the patient-side information processing terminal 3 (for example, personal computer PC). The patient-side information processing terminal 3 is connected to the marker quantification block 1H1A of the effect determination block 1H1 through a signal line SL31. In addition, although not clearly shown in FIG. 13, the patient-side information processing terminal 3 is connected to the medical-institution-side information processing terminal 4 through the network 10 illustrated in FIG. 1.

The sensor 5 for sympathetic nerve marker/parasympathetic nerve marker measurement forms the measuring block 3A on the patient side in FIG. 2, in the same manner as a tensile force sensor, a blood sugar level measuring kit, a blood flow velocity measuring device, and an infrared temperature sensor, which are described later.

The marker quantification block 1H1A has a function of receiving the measured values obtained by the sensor 5 for sympathetic nerve marker/parasympathetic nerve marker measurement through the patient-side information processing terminal 3 and the signal line SL31, and quantifying the measured values so that the measured values can be compared with threshold values.

Numerical values of the markers subjected to the quantification are transmitted to the comparison block 1H1B through a signal line SL32.

The comparison block 1H1B has a function of comparing the numerical values transmitted from the marker quantification block 1H1A with the threshold values. The threshold values are each a numerical value set for determining the effect of the ED treatment for the patient, and are acquired by the comparison block 1H1B from the database 2 illustrated in FIG. 2 through a signal line SL35.

Comparison results obtained by the comparison block 1H1B are transmitted to the treatment result determination block 1H1C through a signal line SL33.

The treatment result determination block 1H1C has a function of determining the treatment result (presence or absence of the therapeutic effect) by examining the presence or absence of improvement in the erectile power and/or ejaculatory ability of the patient from the comparison results obtained by the comparison block 1H1B. As described above, when the numerical value of the sympathetic nerve marker of the patient exceeds the threshold value, it can be determined that the ejaculatory ability has been improved by the therapeutic effect. Meanwhile, when the numerical value of the parasympathetic nerve marker of the patient exceeds the threshold value, it can be determined that the erectile power has been improved by the therapeutic effect.

The treatment result determination block 1H1C executes the determination after the opinion and determination of the medical institution 4 are acquired through a signal line SL34 (bidirectional signal line). However, it is possible to omit the medical institution 4 to omit acquiring the opinion and determination of the medical institution 4.

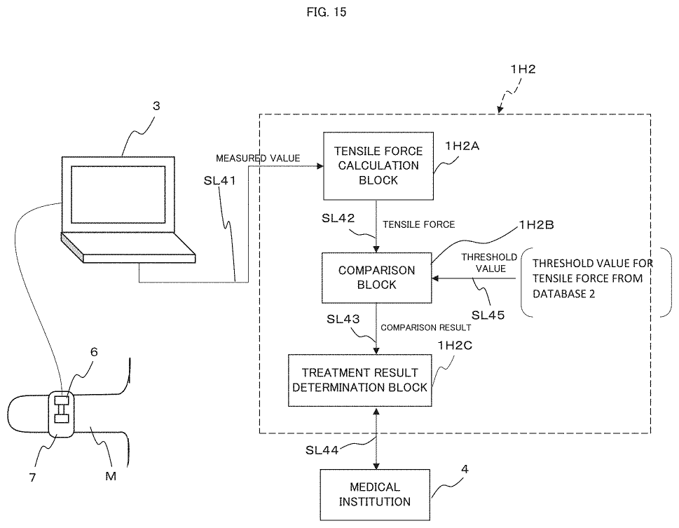

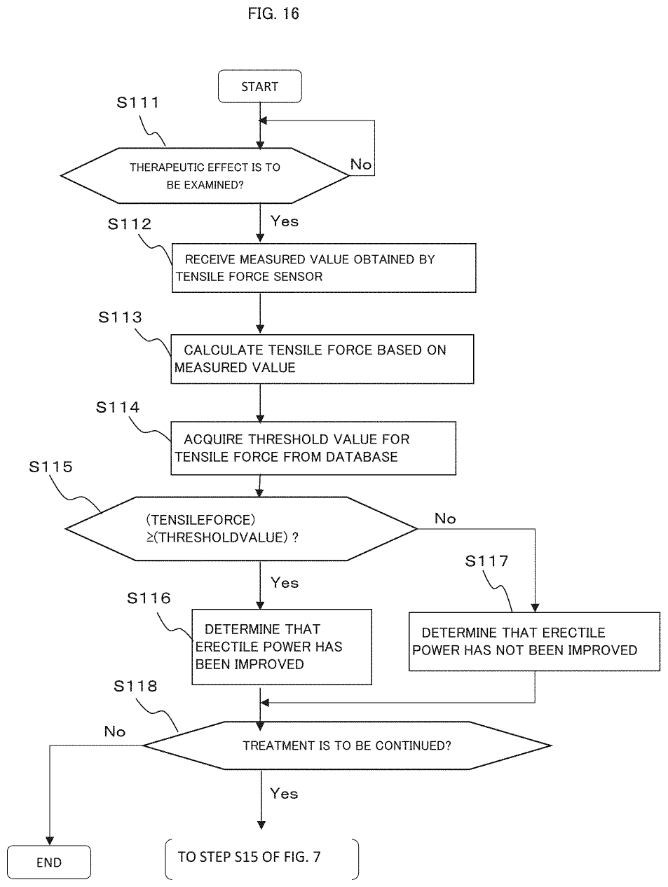

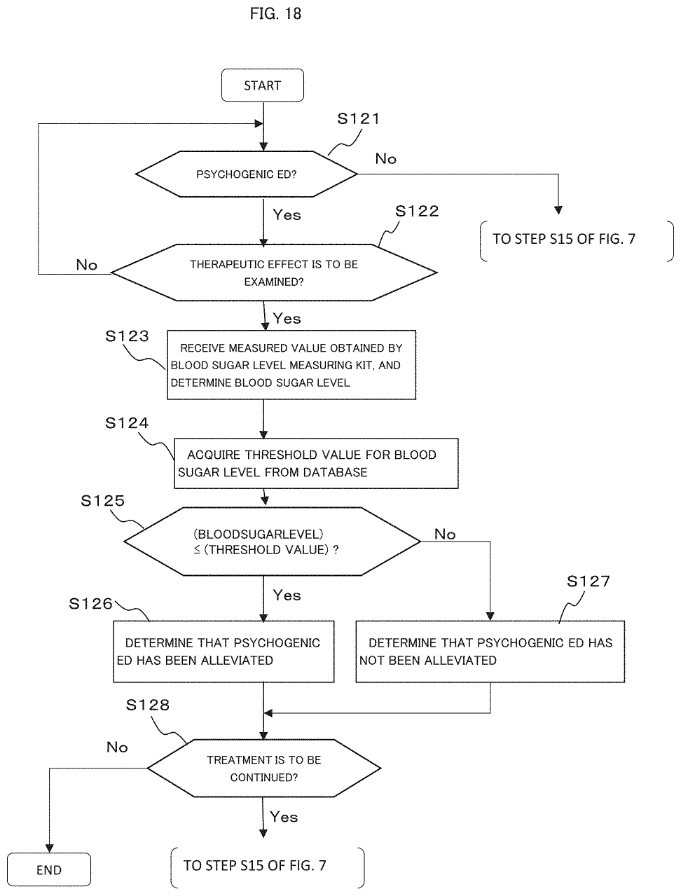

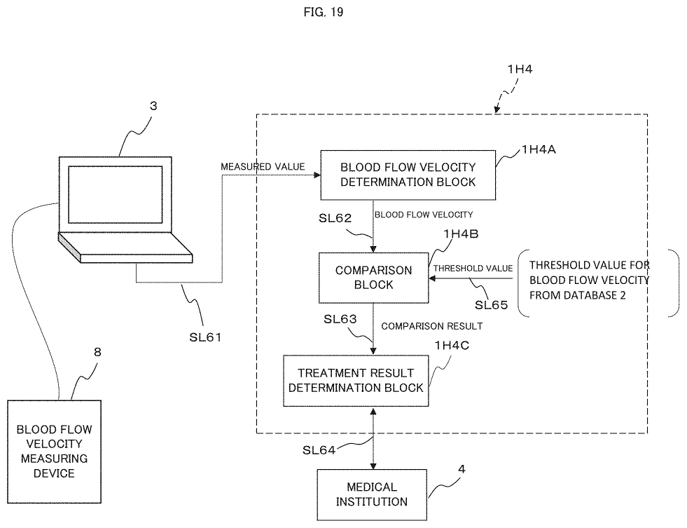

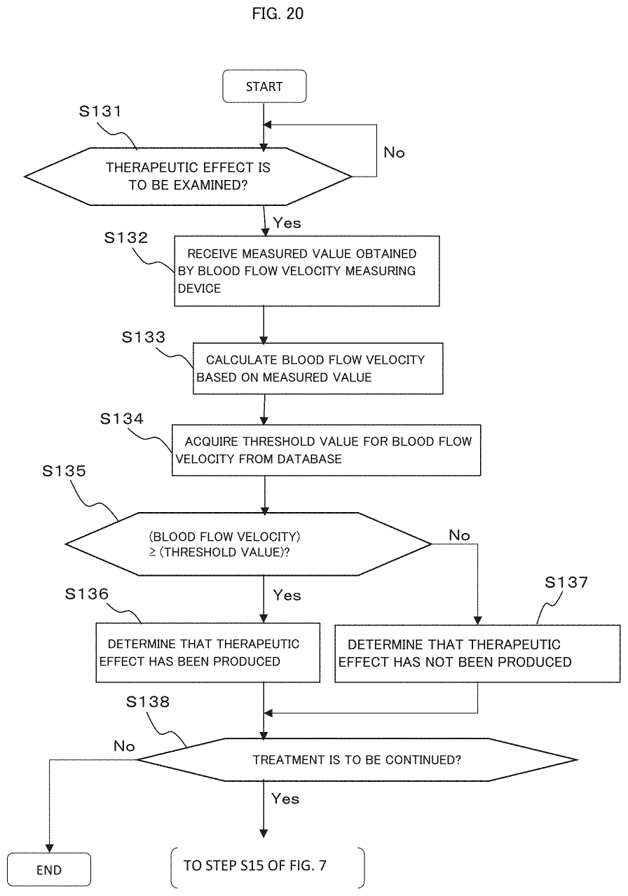

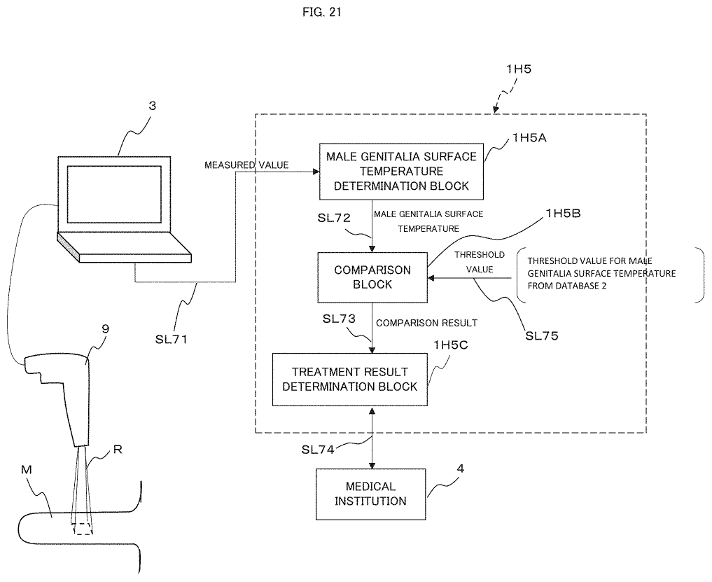

In FIG. 13, reference numeral “4” denotes the “medical institution” itself in place of the “medical-institution-side information processing terminal,” and the same applies to FIG. 15, FIG. 17, FIG. 19, and FIG. 21.

In addition, in the illustrated at least one embodiment, the effect determination block 1H1 using at least a sympathetic nerve marker is formed of a part of the control device 1. However, the effect determination block 1H1 using at least a sympathetic nerve marker can be included in the patient-side information processing terminal 3.

With reference to FIG. 14, a procedure for determining the effect of the ED treatment by the sympathetic nerve marker/parasympathetic nerve marker (sympathetic nerve marker and/or parasympathetic nerve marker) is described.

In FIG. 14, in Step S101, it is determined whether or not it is required to examine the effect of the ED treatment through use of the sympathetic nerve marker/parasympathetic nerve marker.

As a result of the determination of Step S101, when the ED treatment effect is to be examined (“Yes” in Step S101), the process advances to Step S102, and when the ED treatment effect is not to be examined, the process returns to Step S101 (“No” loop in Step S101).

In Step S102, the marker quantification block 1H1A of the effect determination block 1H1 using at least a sympathetic nerve marker receives the measured values obtained by the sensor 5 for sympathetic nerve marker/parasympathetic nerve marker measurement through the patient-side information processing terminal 3 (PC). Then, the marker quantification block 1H1A quantifies the received measured values.

In Step S103, the comparison block 1H1B acquires the threshold values for determining the effect of the ED treatment (for example, threshold value for each of the sympathetic nerve marker and the parasympathetic nerve marker that have been subjected to the quantification) from the database 2.

In Step S104, the comparison block 1H1B compares the measured values quantified in Step S102 (measured value of each of the sympathetic nerve marker and the parasympathetic nerve marker) with the threshold values acquired in Step S103, and determines whether or not the numerical value of any one of the sympathetic nerve marker and the parasympathetic nerve marker exceeds the threshold value for improvement (threshold value at which it is determined that the therapeutic effect has been produced). In this case, the threshold value differs for each individual patient, and differs for each kind of treatment method.

As a result of the determination of Step S104, when the numerical value of the sympathetic nerve marker or the numerical value of the parasympathetic nerve marker exceeds the threshold value for improvement (“Yes” in Step S104), the process advances to Step S105, and when the value is equal to or less than the threshold value for improvement (“No” in Step S104), the process returns to Step S101.

In Step S105 (when the threshold value is exceeded), the comparison block 1H1B determines whether only the numerical value of the sympathetic nerve marker exceeds the threshold value for improvement, only the numerical value of the parasympathetic nerve marker exceeds the threshold value, or the numerical values of both the markers exceed the threshold value.

As a result of the determination of Step S105, when only the numerical value of the sympathetic nerve marker exceeds the threshold value, the process advances to Step S106. When only the numerical value of the parasympathetic nerve marker exceeds the threshold value, the process advances to Step S107. When both the numerical value of the sympathetic nerve marker and the numerical value of the parasympathetic nerve marker exceed the threshold value, the process advances to Step S108.

In Step S106, the treatment result determination block 1H1C determines that the ejaculatory ability of the patient has been improved. In Step S107, the treatment result determination block 1H1C determines that the erectile power of the patient has been improved. In Step S108, the treatment result determination block 1H1C determines that both the ejaculatory ability and the erectile power of the patient have been improved. Then, the process advances to Step S109.

In Step S109, it is determined whether or not to continue the treatment. This determination is performed based on the intention of the patient himself and the determination of the medical institution 4 or the doctor.

As a result of the determination of Step S109, when the treatment is to be continued (“Yes” in Step S109), the process returns to Step S15 of FIG. 7 to continue the treatment.

Meanwhile, as a result of the determination of Step S109, when the treatment is not to be continued (“No” in Step S109), the treatment is ended.

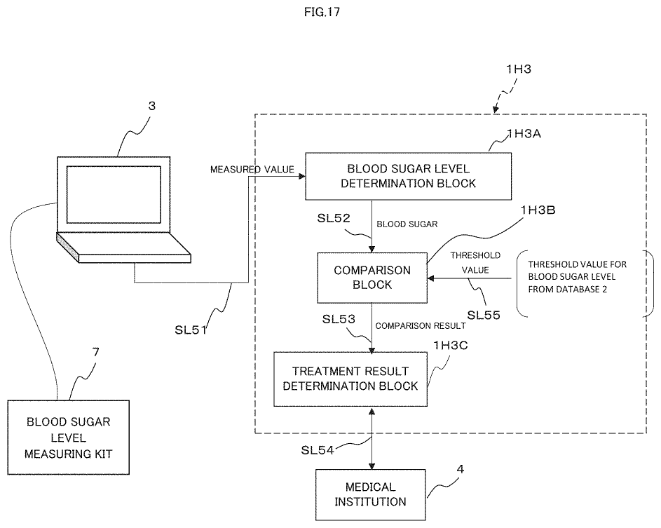

With reference to FIG. 15 and FIG. 16, a mode in which the therapeutic effect is determined through use of a tensile force ascribable to an expansion of a male genitalia in circumferential dimensions at a time of erection is described.

When ED is cured, an erection occurs several times during a sleep. In a case in which a tape-like object is wrapped around a male genitalia (penis), when the male genitalia erects, its circumferential dimension increases, to thereby cause a tensile force to act on the wrapped tape. Through measurement of such a tensile force by using a sensor, the patient's erection and its level can be determined, to thereby be able to estimate the effect of the ED treatment.

In place of such a tensile force sensor, paper of a type that gradually changes in color depending on an applied tension, for example, pressure-sensitive paper, may be used.

FIG. 15 is an illustration of details of an effect determination block 1H2 using a male genitalia tensile force, for determining the therapeutic effect by measuring a tensile force acting on a tape wound around a male genitalia due to an erection. The effect determination block 1H2 using a male genitalia tensile force is responsible for a part of the function of the treatment result determination block 1H illustrated in FIG. 2.

The effect determination block 1H2 using a male genitalia tensile force includes a tensile force calculation block 1H2A, a comparison block 1H2B, and a treatment result determination block 1H2C.

The tensile force sensor 6 is attached to a tape 7 wound around a male genitalia M of the patient, and can measure the tensile force acting on the tape 7 (circumferential direction of the male genitalia M). The tensile force sensor 6 is connected to the patient-side information processing terminal 3, and the patient-side information processing terminal 3 is connected to the tensile force calculation block 1H2A of the effect determination block 1H2 through a signal line SL41. The patient-side information processing terminal 3 can also be connected to the medical-institution-side information processing terminal 4 through the network 10.

The tensile force calculation block 1H2A has a function of receiving a result of the measurement performed by the tensile force sensor 6 through the patient-side information processing terminal 3 and the signal line SL41 and determining the tensile force that has acted on the tensile force sensor 6. The tensile force determined by the tensile force calculation block 1H2A is transmitted to the comparison block 1H2B through a signal line SL42.

The comparison block 1H2B has a function of comparing the tensile force determined by the tensile force calculation block 1H2A with a threshold value for the tensile force (threshold value for determining the effect of the ED treatment), which has been acquired from the database 2 illustrated in FIG. 2 through a signal line SL45.

A result of the comparison is transmitted to the treatment result determination block 1H2C through a signal line SL43.

The treatment result determination block 1H2C has a function of determining the presence or absence of the therapeutic effect by determining whether or not the tensile force of the male genitalia acting on the tape 7 at the time of erection of the patient has been improved from the comparison result obtained by the comparison block 1H2B. That is, when the measured tensile force is equal to or greater than the threshold value, it is determined that the erectile power has been improved and the effect of the ED treatment has been produced.

The treatment result determination block 1H2C executes the determination after the opinion and determination of the medical institution 4 are acquired through a signal line SL44 (bidirectional signal line). However, it is possible to omit acquiring the opinion and determination of the medical institution 4.

With reference to FIG. 16, a procedure for determining the therapeutic effect by measuring the tensile force acting on the tape 7 by the tensile force sensor 6 is described.

In Step S111 of FIG. 16, it is determined whether or not to examine the therapeutic effect by measuring the tensile force acting on the tape 7 at the time of erection. When the therapeutic effect is to be examined (“Yes” in Step S111), the process advances to Step S112, and when the therapeutic effect is not to be examined, the process returns to Step S111 (“No” loop in Step S111).

In Step S112, the tensile force calculation block 1H2A of the effect determination block 1H2 using a male genitalia tensile force receives the measured value obtained by the tensile force sensor 6 through the patient-side information processing terminal 3 (PC). Then, the tensile force calculation block 1H2A calculates the tensile force that has acted on the tape 7 (Step S113).

In Step S114, the comparison block 1H2B acquires the threshold value for the tensile force from the database 2. Then, the comparison block 1H2B compares the calculated tensile force with the threshold value acquired in Step S114 (Step S115).

In Step S115, when the tensile force is equal to or greater than the threshold value (“Yes” in Step S115), the process advances to Step S116, and when the tensile force is not equal to or greater than the threshold value (“No” in Step S115), the process advances to Step S117.

In Step S116 (when the tensile force is equal to or greater than the threshold value), the treatment result determination block 1H2C determines that the erectile power of the patient has been improved. Meanwhile, in Step S117 (when the tensile force is not equal to or greater than the threshold value), the treatment result determination block 1H2C determines that the erectile power of the patient has not been improved.

In Step S118, it is determined whether or not to continue the treatment. This determination is performed based on the intention of the patient himself and the determination of the medical institution 4 or the doctor.

As a result of the determination of Step S118, when the treatment is to be continued (“Yes” in Step S118), the process returns to Step S15 of FIG. 7 to continue the treatment including the change to another therapy. Meanwhile, when the treatment is not to be continued (“No” in Step S118), the treatment is ended.

With reference to FIG. 17 and FIG. 18, a mode in which a psychotherapeutic effect is determined based on reduction of a blood sugar level is described.

It has been confirmed that a blood sugar level of an ED patient is lowered when the ED patient receives psychotherapy. In addition, diabetes causes damage to blood vessels and nerves, and is therefore a disease having the highest rate of causing ED among lifestyle-related diseases, and hence alleviation of ED can be expected by lowering the blood sugar level. Therefore, the effect of the ED treatment can be estimated by measuring the blood sugar level of the patient.

FIG. 17 is an illustration of details of an effect determination block 1H3 using a blood sugar level, for determining the therapeutic effect by measuring the blood sugar level. The effect determination block 1H3 is responsible for a part of the function of the treatment result determination block 1H illustrated in FIG. 2.

The effect determination block 1H3 using a blood sugar level includes a blood sugar level determination block 1H3A, a comparison block 1H3B, and a treatment result determination block 1H3C.

A blood sugar level measuring kit 7 is connected to the patient-side information processing terminal 3, and the patient-side information processing terminal 3 is connected to the blood sugar level determination block 1H3A of the effect determination block 1H3 through a signal line SL51. In addition, the patient-side information processing terminal 3 is connected to the medical-institution-side information processing terminal 4 through the network 10 illustrated in FIG. 1.

The blood sugar level determination block 1H3A has a function of receiving the measured value obtained by the blood sugar level measuring kit 7 through the patient-side information processing terminal 3 and the signal line SL51, and determining the blood sugar level. The determined blood sugar level is transmitted to the comparison block 1H3B through a signal line SL52.

The comparison block 1H3B has a function of comparing the blood sugar level transmitted from the blood sugar level determination block 1H3A with a threshold value for the blood sugar level, which has been acquired from the database 2 illustrated in FIG. 2 through a signal line SL55. A result of the comparison is transmitted to the treatment result determination block 1H3C through a signal line SL53.

The treatment result determination block 1H3C has a function of determining the treatment result (presence or absence of the therapeutic effect) from the comparison result obtained by the comparison block 1H3B. That is, when the blood sugar level of the patient is equal to or less than the threshold value, it is determined that the blood sugar level has been lowered and the ED has been alleviated.

The treatment result determination block 1H3C executes the determination after the opinion and determination of the medical institution 4 are acquired through a signal line SL54 (bidirectional signal line). However, it is possible to omit acquiring the opinion and determination of the medical institution 4.

FIG. 18 is an illustration of a procedure for determining the therapeutic effect by measuring (lowering of) the blood sugar level of the patient by the blood sugar level measuring kit 7.

The procedure of FIG. 18 is substantially the same as that of FIG. 16 except that: the parameter is the blood sugar level; when the blood sugar level of the patient is equal to or less than the threshold value, it is determined that the blood sugar level has been lowered and the ED has been alleviated; and the alleviation of the ED is determined but the improvement in the erectile power is not determined. Step S121 to Step S128 of FIG. 18 correspond to Step S111 to Step S118 of FIG. 16.

Next, with reference to FIG. 19 and FIG. 20, a mode in which the therapeutic effect is determined based on a blood flow velocity is described.

The male genitalia having a strong erectile power is satisfactory in blood flow, and the blood flow in, for example, the fingers of that person's hand is also satisfactory. Therefore, when the blood flow of the patient is checked and the blood flow velocity is measured, it is possible to, for example, examine the erectile power and estimate the effect of the ED treatment.

FIG. 19 is an illustration of details of an effect determination block 1H4 using a blood flow velocity, for determining the therapeutic effect by measuring the blood flow velocity. The effect determination block 1H4 is responsible for a part of the function of the treatment result determination block 1H illustrated in FIG. 2.

The effect determination block 1H4 using a blood flow velocity includes a blood flow velocity determination block 1H4A, a comparison block 1H4B, and a treatment result determination block 1H4C.

A blood flow velocity measuring device 8 is connected to the patient-side information processing terminal 3, and the patient-side information processing terminal 3 is connected to the blood flow velocity determination block 1H4A of the effect determination block 1H4 through a signal line SL61. In addition, the patient-side information processing terminal 3 is connected to the medical-institution-side information processing terminal 4 through the network 10 illustrated in FIG. 1.

The blood flow velocity determination block 1H4A has a function of receiving the measured value obtained by the blood flow velocity measuring device 8 through the patient-side information processing terminal 3 and the signal line SL61, and determining the blood flow velocity. The determined blood flow velocity is transmitted to the comparison block 1H4B through a signal line SL62.

The comparison block 1H4B has a function of comparing the blood flow velocity transmitted from the blood flow velocity determination block 1H4A with a threshold value for the blood flow velocity, which has been acquired from the database 2 illustrated in FIG. 2 through a signal line SL65. A result of the comparison is transmitted to the treatment result determination block 1H4C through a signal line SL63.

The treatment result determination block 1H4C has a function of determining the treatment result (presence or absence of the therapeutic effect) from the comparison result obtained by the comparison block 1H4B, and when the blood flow velocity of the patient is equal to or greater than the threshold value, the treatment result determination block 1H4C determines that the erectile power has been improved and the effect of the ED treatment has been produced.

The treatment result determination block 1H4C executes the determination after the opinion and determination of the medical institution 4 are acquired through a signal line SL64 (bidirectional signal line). However, it is possible to omit acquiring the opinion and determination of the medical institution 4.

FIG. 20 is an illustration of a procedure for determining the therapeutic effect by measuring the blood flow velocity of the patient by the blood flow velocity measuring device 8.

The procedure of FIG. 20 is substantially the same as those of FIG. 16 and FIG. 18 except that: the parameter is the blood flow velocity; when the blood flow velocity is equal to or greater than the threshold value, it is determined that the erectile power has been improved and the effect of the ED treatment has been produced; and the presence or absence of the therapeutic effect is determined. Step S131 to Step S138 of FIG. 20 correspond to Step S111 to Step S118 of FIG. 16 and Step S121 to Step S128 of FIG. 18.

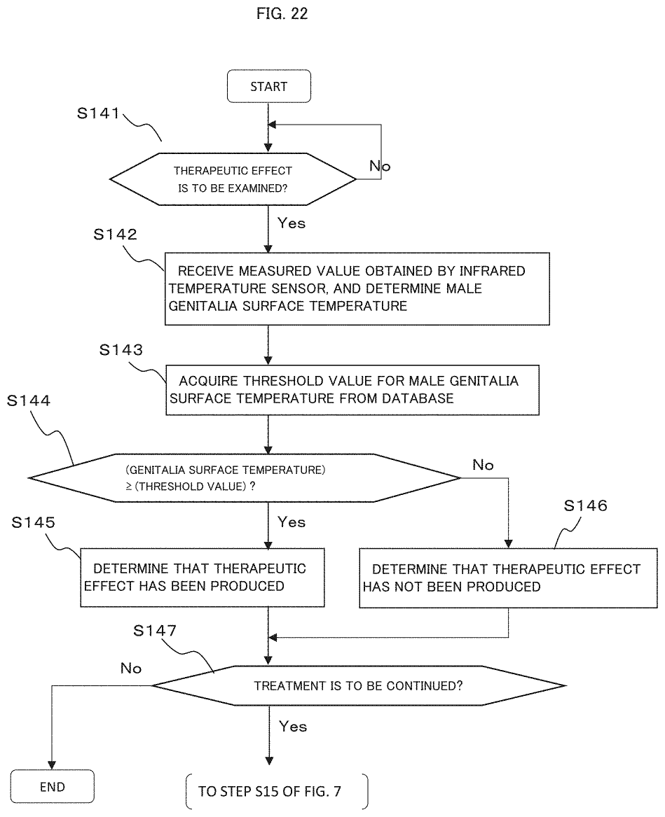

With reference to FIG. 21 and FIG. 22, a mode in which the therapeutic effect is determined based on a male genitalia surface temperature is described.

According to the inventors' research, when a male genitalia erects, the male genitalia surface temperature rises. Therefore, when the male genitalia surface temperature of the patient can be measured to confirm the temperature rise, it can be estimated that the erectile power has been improved and the effect of the ED treatment has been produced.

FIG. 21 is an illustration of details of an effect determination block 1H5 using a male genitalia surface temperature, for determining the therapeutic effect by measuring the male genitalia surface temperature. The effect determination block 1H5 is responsible for a part of the function of the treatment result determination block 1H illustrated in FIG. 2.

The effect determination block 1H5 using a male genitalia surface temperature includes a male genitalia surface temperature determination block 1H5A, a comparison block 1H5B, and a treatment result determination block 1H5C.

In the illustrated at least one embodiment, an infrared temperature sensor 9 is used as a device for measuring the male genitalia surface temperature, and the surface of the male genitalia M is irradiated with infrared rays R to measure the surface temperature. However, it is also possible to perform the measurement by attaching temperature-sensitive paper to the surface of the male genitalia.

The infrared temperature sensor 9 is connected to the patient-side information processing terminal 3, and the patient-side information processing terminal 3 is connected to the male genitalia surface temperature determination block 1H5A of the effect determination block 1H5 through a signal line SL71. In addition, the patient-side information processing terminal 3 is connected to the medical-institution-side information processing terminal 4 through the network 10 illustrated in FIG. 1.

The male genitalia surface temperature determination block 1H5A has a function of receiving the measured value obtained by the infrared temperature sensor 9 through the patient-side information processing terminal 3 and the signal line SL71, and determining the male genitalia surface temperature. The determined male genitalia surface temperature is transmitted to the comparison block 1H5B through a signal line SL72.

The comparison block 1H5B has a function of comparing the male genitalia surface temperature transmitted from the male genitalia surface temperature determination block 1H5A with a threshold value for the male genitalia surface temperature acquired from the database 2 illustrated in FIG. 2 through a signal line SL75. A result of the comparison is transmitted to the treatment result determination block 1H5C through a signal line SL73.

The treatment result determination block 1H5C has a function of determining the treatment result (presence or absence of therapeutic effect) from the comparison result obtained by the comparison block 1H5B, and determining that, when the male genitalia surface temperature of the patient is equal to or greater than the threshold value, the erectile power has been improved and the effect of the ED treatment has been produced.

The treatment result determination block 1H5C executes the determination after the opinion and determination of the medical institution 4 are acquired through a signal line SL74 (bidirectional signal line). However, it is possible to omit acquiring the opinion and determination of the medical institution 4.

With reference to FIG. 22, a procedure for determining the therapeutic effect by measuring the male genitalia surface temperature of the patient is described.

In Step S141, it is determined whether or not to examine the therapeutic effect by measuring the male genitalia surface temperature of the patient by the infrared temperature sensor 9. When the therapeutic effect is to be examined (“Yes” in Step S141), the process advances to Step S142, and when the therapeutic effect is not to be examined, the process returns to Step S141 (“No” loop in Step S141).

In Step S142, the male genitalia surface temperature determination block 1H5A of the effect determination block 1H5 using a male genitalia surface temperature receives the measured value obtained by the infrared temperature sensor 9, and determines the male genitalia surface temperature.

In Step S143, the comparison block 1H5B acquires the threshold value for the male genitalia surface temperature from the database 2. Then, the comparison block 1H5B compares the male genitalia surface temperature of the patient with the threshold value for the male genitalia surface temperature, which is acquired in Step S143 (Step S144). When the male genitalia surface temperature is equal to or greater than the threshold value (“Yes” in Step S144), the process advances to Step S145, and when the male genitalia surface temperature is not equal to or greater than the threshold value (“No” in Step S144), the process advances to Step S146.

In Step S145 (when the male genitalia surface temperature rises to the threshold value or more), the treatment result determination block 1H5C determines that the effect of the ED treatment has been produced. Meanwhile, in Step S146 (when the male genitalia surface temperature is not equal to or greater than the threshold value), the treatment result determination block 1H5C determines that the effect of the ED treatment has not been produced.

In Step S147, it is determined whether or not to continue the treatment. This determination is performed based on the intention of the patient himself and the determination of the medical institution 4 or the doctor. When the treatment is to be continued (“Yes” in Step S147), the process returns to Step S15 of FIG. 7 to continue the treatment including the change to another therapy. Meanwhile, when the treatment is not to be continued (“No” in Step S147), the treatment is ended.

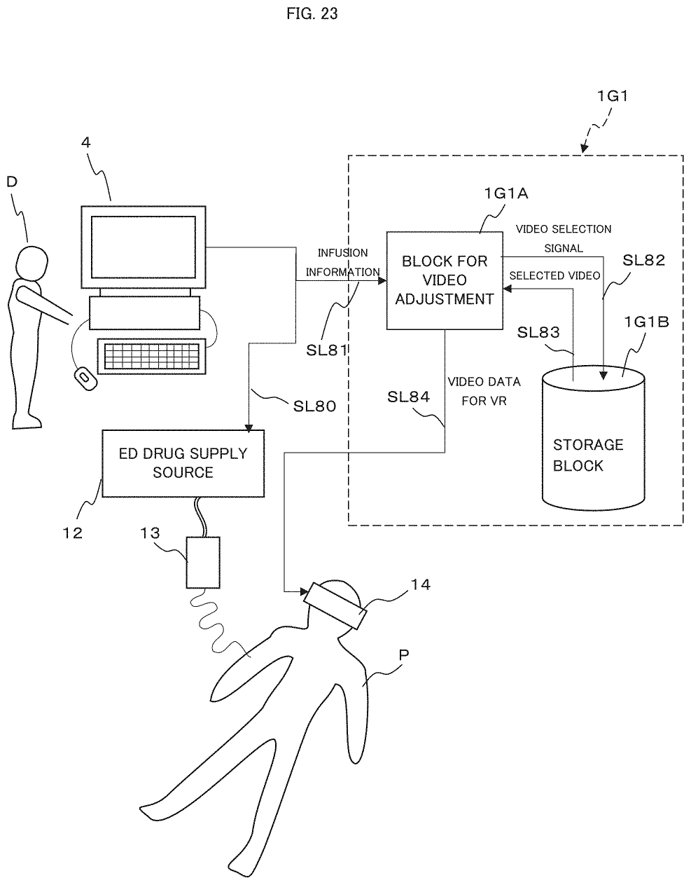

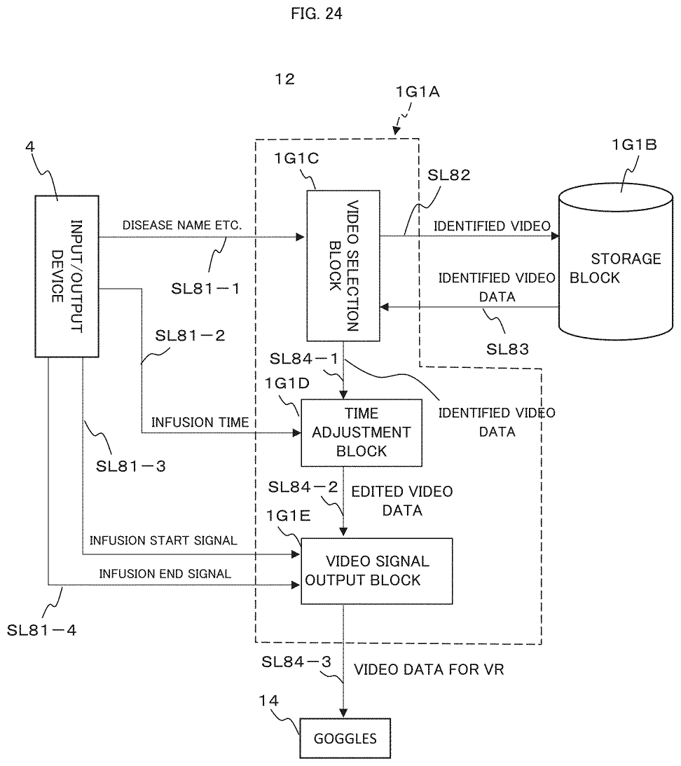

Next, with reference to FIG. 23 and FIG. 24, one mode of the treatment of organic ED in the illustrated at least one embodiment is described.

In FIG. 23, an organic block 1G1 is responsible for a part of the function of the organic block 1G of FIG. 2.

In FIG. 23, while a drug for the treatment of organic ED which is injected by a drip is being administered by infusion, a VR moving image (or video) in which the drug is reaching the male genitalia or an organ relating to an erectile function (for example, organ of an androgen secretion system) is being presented to the patient by VR goggles. In this case, the video can be accompanied by a voice.

According to the inventors' experiment, patients who watch the above-mentioned moving image or video are significantly improved in efficiency of ED treatment as compared with patients who do not watch the moving image or video. It is estimated that the improvement is ascribable to psychogenic effects (so-called “placebo effect” and others).

In FIG. 23, the organic block 1G1 includes a block 1G1A for video adjustment and a storage block 1G1B.

A doctor D or the like at the medical institution operates an input/output device 4 serving as a medical-institution-side information processing terminal to transmit a control signal to an ED drug supply source 12 through a signal line SL80, and uses a drip container 13 to perform infusion administration on a patient P in a consultation room. The doctor D or the like also operates the input/output device 4 to transmit infusion information, which includes the patient's disease name, a time period for performing infusion (infusion time period), an infusion start instruction, and an infusion end instruction, to the block 1G1A for video adjustment of the organic block 1G1 through a signal line SL81.

In this case, in place of the doctor D and the like and the input/output device 4, the ED drug supply source 12 and the drip container 13 can be connected to the control device 1 illustrated in FIG. 2.

The patient P lying in the consultation room wears goggles 14 for VR.

The block 1G1A for video adjustment of the organic block 1G1 has a function of selecting and identifying an optimum video (or moving image) to be provided to (the goggles 14 for VR of) the patient P based on the infusion information acquired from the input/output device 4 (or control device 1), and transmitting a video selection signal (selected video request signal) for requesting the selected video through a signal line SL82 from the storage block 1G1B in which various videos are stored.

The block 1G1A for video adjustment of the organic block 1G1 also has a function of acquiring video data for VR from the storage block 1G1B through a signal line SL83 based on the selected video, and providing the video data for VR to the goggles 14 for VR worn by the patient through a signal line SL84.

The storage block 1G1B stores various videos, images, and voices that are effective during the ED treatment. Examples of such videos, images, and voices include a video of the drug reaching the male genitalia or the organ relating to the erectile function (for example, organ of the androgen secretion system) and voice for explanation of the video. Then, the block 1G1A for video adjustment selects an optimum video from among such videos, images, and voices depending on, for example, the situation of the patient's ED.

In FIG. 24, which is a more detailed functional block diagram, the block 1G1A for video adjustment includes a video selection block 1G1C, a time adjustment block 1G1D, and a video signal output block 1G1E.

With reference to FIG. 24, the function of the organic block 1G1 for providing a VR video to the patient is described in more detail.

The video selection block 1G1C acquires information including the patient's disease name and specific medical condition from the input/output device 4 through a signal line SL81-1, and selects and identifies the optimum video to be provided to (the goggles 14 for VR of) the patient P based on the acquired information.

In addition, the video selection block 1G1C requests (transmits a video request signal to) the storage block 1G1B for the selected and identified optimum video through the signal line SL82, and acquires the selected and identified video data from the storage block 1G1B through a signal line SL83.

The selected and identified video data acquired by the video selection block 1G1C is transmitted to the time adjustment block 1G1D through a signal line SL84-1.

The time adjustment block 1G1D edits, based on the infusion time (time period for performing infusion administration on the patient P, which is determined in advance by the doctor D or the like) transmitted from the input/output device 4 through a signal line SL81-2, the video data transmitted from the video selection block 1G1C in accordance with the infusion time, and, for example, edits the video data so that its main part falls within the infusion time.

The video data edited by the block 1G1A for video adjustment is transmitted to the video signal output block 1G1E through a signal line SL84-2. The video signal output block 1G1E transmits, based on an infusion start signal for instructing to start the infusion, the edited video data for VR transmitted from the block 1G1A for video adjustment to the goggles 14 for VR worn by the patient through a signal line SL84-3.

In addition, the video signal output block 1G1E ends transmission of the video data for VR to the goggles 14 for VR worn by the patient based on an infusion end signal for instructing to end the infusion.

In the organic block 1G1 illustrated in FIG. 23 and FIG. 24, the video data for VR having the above-mentioned content is being presented to the patient by the goggles 14 while the drug for the treatment of organic ED is being administered to the patient by infusion. Therefore, the efficiency of the ED treatment is significantly improved. It is also effective for the video to be accompanied by the voice and music for explanation when the VR moving image is being presented.

FIG. 23 and FIG. 24 are the illustrations of an example of the treatment of the organic ED, but the improvement of the effect of the ED treatment through use of images presented in VR has been confirmed not only for organic ED but also for the psychogenic ED and the mixed ED. Therefore, the equipment of FIG. 23 and FIG. 24 may be provided not only to the organic block 1G1 but also to the psychogenic-or-mixed block 1F of FIG. 2.

According to the illustrated at least one embodiment, the ED patient can contact a medical institution including a doctor through the patient-side information processing terminal 3 and the network 10 (for example, the Internet), and it is required for the patient to visit a specialized medical institution and undergo the treatment face-to-face with the doctor only for minimum purposes including a prescription. Therefore, the psychological resistance of the ED patient to privacy issues in, for example, undergoing the treatment face-to-face with the doctor, is reduced, to thereby reduce a probability of dropping out during the treatment.

That is, in the illustrated at least one embodiment, the requirement for the patient to visit the medical institution or undergo the treatment face-to-face with the doctor is minimized, and the fact that the patient is “undergoing ED treatment” is kept secret as requested by the patient. Therefore, the ED patient's sense of resistance to the treatment can be greatly reduced. Then, the patient no longer has resistance to the use of the PDE5 inhibitor in combination with the psychotherapy such as cognitive behavioral therapy, and its consultation rate is expected to be improved.

Further, according to the illustrated at least one embodiment, the cognitive behavioral therapy can be performed at the time of performing the ED treatment, and hence not only the treatment involving the administration of the PDE5 inhibitor but also the psychotherapy including the cognitive behavioral therapy strongly recommended by specialists can be used in combination.

It is to be understood that it is also possible to execute the treatment involving the administration of the PDE5 inhibitor by, for example, a procedure for issuing a prescription for the PDE5 inhibitor, in combination with the psychotherapy or another therapy.

In the illustrated at least one embodiment, it is possible to select various kinds of cognitive behavioral therapies depending on the situation of the patient by setting the cognitive behavioral therapy for rewriting the patient's cognition in terms of the ED itself and other therapies, to thereby be able to expect the effective treatment. Then, the patient himself faces the real psychological factors or the deep psychological factors, further faces the fact of having ED itself, and rewrites his cognitive distortion, to thereby be able to resolve the psychological factors in the ED.

In addition thereto, in the illustrated at least one embodiment, after the cognitive behavioral therapy is executed, the follow-up observation for confirming the effect of the treatment is executed, and hence it is expected to efficiently increase the therapeutic effect by changing the therapy depending on the effect of the treatment.

Further, the medical-institution-side information processing terminal 4 to be used on the medical institution side, for example, by the doctor, is connected through the network 10, and hence the situation of the patient, which is updated every moment, can be examined on the medical institution side, for example, by the doctor.

It should be added that the illustrated at least one embodiment is merely an example, and is not the description intended to limit the technical scope of the present invention.

For example, in the illustrated at least one embodiment, the psychotherapy is exemplified by the cognitive behavioral therapy, but other kinds of psychotherapy can also be applied.

What is claimed is:

1. A system for a patient with erectile dysfunction, comprising:
a control device;
an information processing terminal to be used by the patient;
a database configured to store information on the patient and information relating to an erectile dysfunction treatment; and
an information processing terminal to be used on a medical institution side,
wherein the control device has a function of selecting information relating to therapy for resolving the erectile dysfunction based on the information on the patient and the information relating to the erectile dysfunction treatment.

2. A method of using an information processing system for a patient with erectile dysfunction, the method comprising:
transmitting a situation relating to the erectile dysfunction of the patient from a patient-side information processing terminal to a control device;
determining, by the control device, information relating to an erectile dysfunction treatment in response to the received situation of the patient; and
transmitting the determined information relating to the erectile dysfunction treatment to the patient-side information processing terminal.

3. A program for executing the following processes for a patient with erectile dysfunction, the processes comprising:
transmitting a situation relating to the erectile dysfunction of the patient from a patient-side information processing terminal to a control device;
determining, by the control device, information relating to an erectile dysfunction treatment in response to the situation of the patient; and
transmitting the determined information relating to the erectile dysfunction treatment to the patient-side information processing terminal.
